(19) United States
(12) Patent Application Publication
(10) Pub. No.: US 2025/0032545 A1
(43) Pub. Date: Jan. 30, 2025

(54) MIR155 AS THE INTEGRATION SITE TO OVERCOME ACUTE GRAFT VERSUS HOST DISEASE OF GENE-MODIFIED IMMUNE EFFECTOR CELLS

(71) Applicants: Ohio State Innovation Foundation, Columbus, OH (US); Nationwide Children's Hospital, Columbus, OH (US)

(72) Inventors: Parvathi Ranganathan, Columbus, OH (US); Meisam Naeimi Kararoudi, Columbus, OH (US); Yasemin Sezgin, Columbus, OH (US); Dean Lee, Columbus, OH (US)

(21) Appl. No.: 18/785,173

(22) Filed: Jul. 26, 2024

Related U.S. Application Data
(60) Provisional application No. 63/515,654, filed on Jul. 26, 2023.

(51) Int. Cl. A61K 35/17; A61K 39/00; C07K 16/28; C07K 16/30; C12N 5/00; C12N 5/0783; C12N 9/22; C12N 15/11; C12N 15/86

(52) U.S. Cl. CPC A61K 35/17; A61K 39/4611; A61K 39/4631; A61K 39/464412; A61K 39/464471; C07K 16/2803; C07K 16/3084; C12N 5/0018; C12N 5/0636; C12N 9/22; C12N 15/11; C12N 15/86; A61K 2239/13; C12N 2310/20; C12N 2501/2302; C12N 2501/2307; C12N 2501/2315; C12N 2750/14143

(57) ABSTRACT

The present disclosure relates to CAR-T cells and uses thereof. Disclosed herein is a genetically modified T cell comprising a nucleic acid sequence encoding a chimeric antigen receptor (CAR) polypeptide comprising a single-chain variable fragment (scFV) that specifically binds to a target molecule, wherein the nucleic acid sequence encoding the CAR polypeptide is integrated into an integration site located at a miR-155 host gene.

### CROSS-REFERENCE TO RELATED APPLICATION

This application claims priority to, and the benefit of, U.S. Provisional Patent Application No. 63/515,654, filed Jul. 26, 2023, which is incorporated by reference herein in its entirety.

### STATEMENT REGARDING FEDERALLY SPONSORED RESEARCH

This invention was made with government support under Grant No. R01CA252469 and R01HL163849 awarded by the National Institutes of Health. The government has certain rights in the invention.

### REFERENCE TO SEQUENCE LISTING

The sequence listing submitted on Jul. 26, 2024, as an .XML file entitled “103361-561US1-ST26” created on Jul. 25, 2024, and having a file size of 32,2176 bytes is hereby incorporated by reference pursuant to 37 C.F.R. § 1.52 (e) (5).

### FIELD

The present disclosure relates to genetically modified cells and uses thereof.

### BACKGROUND

More than 8,000 patients receive an allo-hematopoietic stem cell transplant (HSCT) annually in the U.S. alone as a treatment for hematologic malignancies and other primary bone marrow disorders. However, the major barrier for the success of allo-HSCT is the high incidence of acute graft-versus-host disease (aGVHD) and its associated morbidity and mortality. What is needed are new compositions and methods for treating cancer without causing aGVHD.

### SUMMARY

Deletion of miR155 in T cells before transplant can overcome aGvHD. Targeting of miR155 locus as an insertion site can improve cell therapy in combination with CAR for cancers (e.g., blood cancers). This has a distinct advantage of simultaneous miR155 knock-out to overcome GvHD and a site directed CAR DNA insertion for redirected targeting.

Accordingly, in some aspects, disclosed herein are compositions and methods for creating genetically modified T cells and uses thereof for treatment of cancer. The compositions and methods disclosed herein can reduce the risk of acute graft-versus-host disease.

In some aspects, disclosed herein is a genetically modified T cell comprising a nucleic acid sequence encoding a chimeric antigen receptor (CAR) polypeptide comprising a single-chain variable fragment (scFV) that specifically binds to a target molecule, wherein the nucleic acid sequence encoding the CAR is integrated into an integration site located at a miR-155 host gene.

In some embodiments, the integration site is at exon 1, exon 2, exon 3, or the transcriptional start site of the miR-155 host gene.

In some embodiments, the genetically modified cell further comprises ribonucleoprotein (RNP) complex comprising a CRISPR/Cas endonuclease (Cas9) system complexed with one or more guide RNAs targeting the miR-155 host gene or a fragment thereof; and an AAV vector comprising a plasmid, nucleic acid, or construct comprising a polynucleotide sequence encoding the CAR polypeptide; wherein the polynucleotide sequence is flanked by homology arms.

In some embodiments, the homology arm comprises a sequence at least 80% identical to SEQ ID NO: 3 or 4, or a fragment thereof.

In some embodiments, the CRISPR/Cas9 system comprises a first guide RNA and a second guide RNA. In some embodiments, the first guide RNA comprises a polynucleotide sequence at least 80% identical to SEQ ID NO: 1 or a fragment thereof. In some embodiments, the second guide RNA comprises a polynucleotide sequence at least 80% identical to SEQ ID NO: 2 or a fragment thereof.

The target molecule of the CAR can be a cancer-related protein (including, for example, CD19, GD2, or CD33).

In some embodiments, the AAV vector comprises a polynucleotide sequence at least 80% identical to SEQ ID NO: 6 or a fragment thereof. In some embodiments, the T cell is a primary T cell, a T cell line, a tumor infiltrating lymphocyte, an effector T cell, a memory T cell, a TEMRA, or a stem cell-like memory T cell.

In some aspects, disclosed herein is a method for treating cancer in a subject, comprising administering to the subject a therapeutically effective amount of the genetically modified T cell of any preceding aspect. The T cell can be cultured ex vivo for at least 2 days prior to the administration. In some embodiments, the T cell is cultured with IL-2, IL-7, or IL-15, or any combination thereof. In some embodiments, the T cell is derived from the subject or a different subject. Administration of the genetically modified T cell can prevent and/or treat acute graft-versus-host disease.

Also disclosed herein is a gene-editing system for engineering a CAR-T cell, said system comprising a ribonucleoprotein (RNP) complex comprising a CRISPR/Cas endonuclease (Cas9) system complexed with one or more guide RNAs targeting a miR-155 host gene or a fragment thereof; and an AAV vector comprising a plasmid, nucleic acid, or construct comprising a polynucleotide sequence encoding the CAR polypeptide; wherein the polynucleotide sequence is flanked by homology arms.

Also disclosed herein is a method of creating a genetically modified T cell comprising a nucleic acid sequence encoding a chimeric antigen receptor (CAR) polypeptide integrated into an integration site located at a miR-155 host gene, said method comprising obtaining a T cell; and introducing into the T cell the gene-editing system of any preceding aspect thereby creating the genetically modified T cell.

Also disclosed herein is a method for treating cancer in a subject, comprising creating a genetically modified T cell using the method of any preceding aspect; and administering to the subject a therapeutically effective amount of the genetically modified T cell.

### BRIEF DESCRIPTION OF THE DRAWINGS

The accompanying figures, which are incorporated in and constitute a part of this specification, illustrate several aspects described below.

FIG. 1 shows the generation of CRISPR miR155-KO T cells human primary T cells.

FIG. 2A shows the generation of CRISPR miR155-KO/CD33CAR T cells human primary T cells.

FIG. 2B shows CAR staining on Day 2 post CAR-T generation of CRISPR miR155-KO/CD33CAR T cells human primary T cells.

FIG. 2C shows CAR staining on Day 7 post CAR-T generation of CRISPR miR155-KO/CD33CAR T cells human primary T cells.

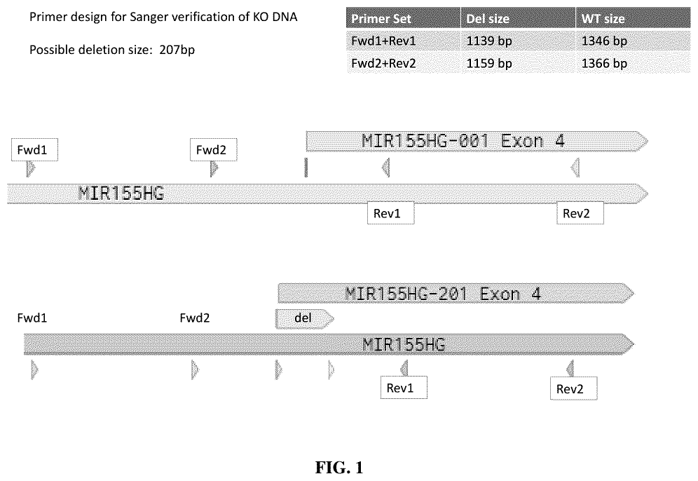

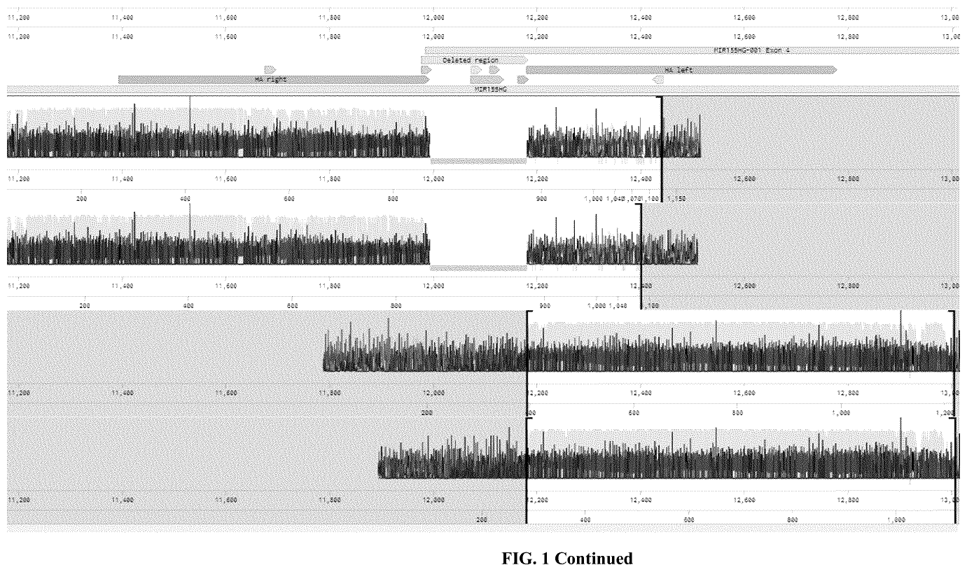

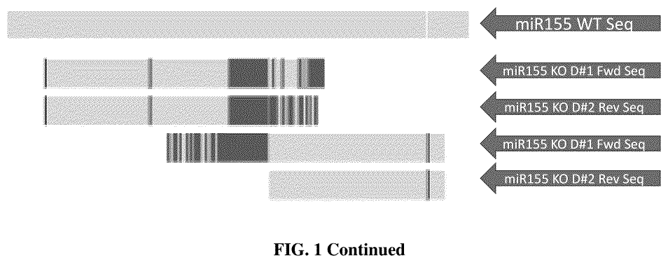

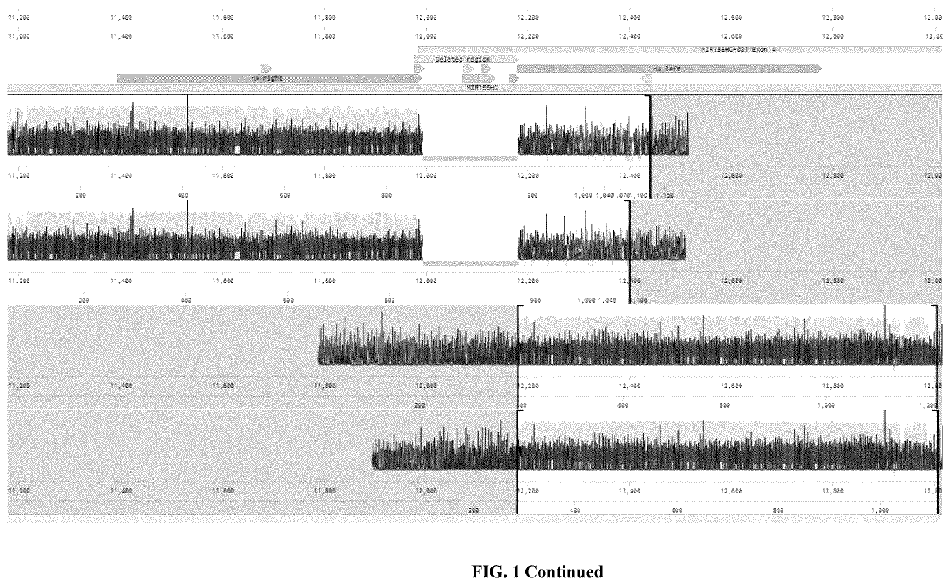

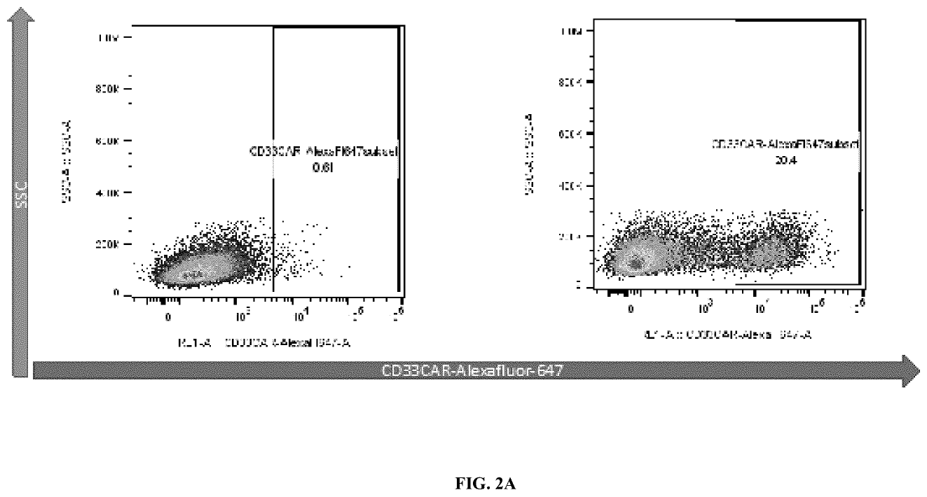

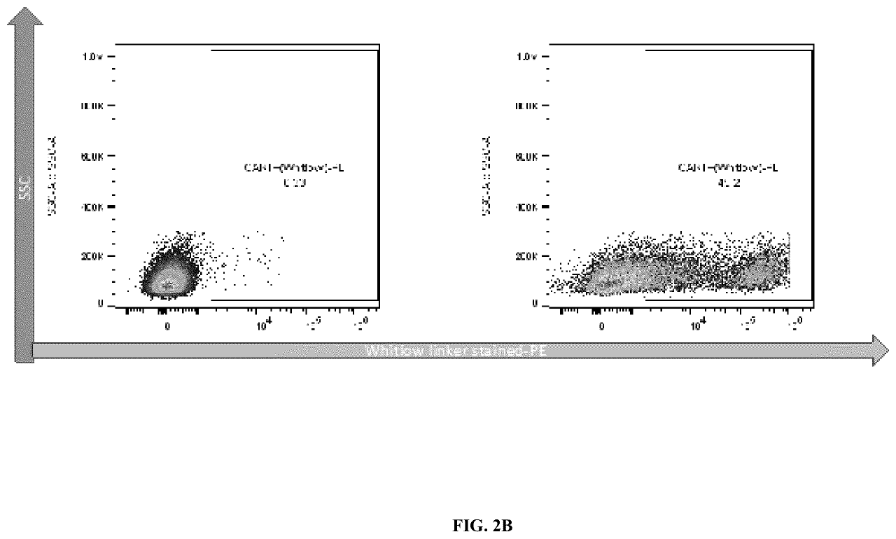

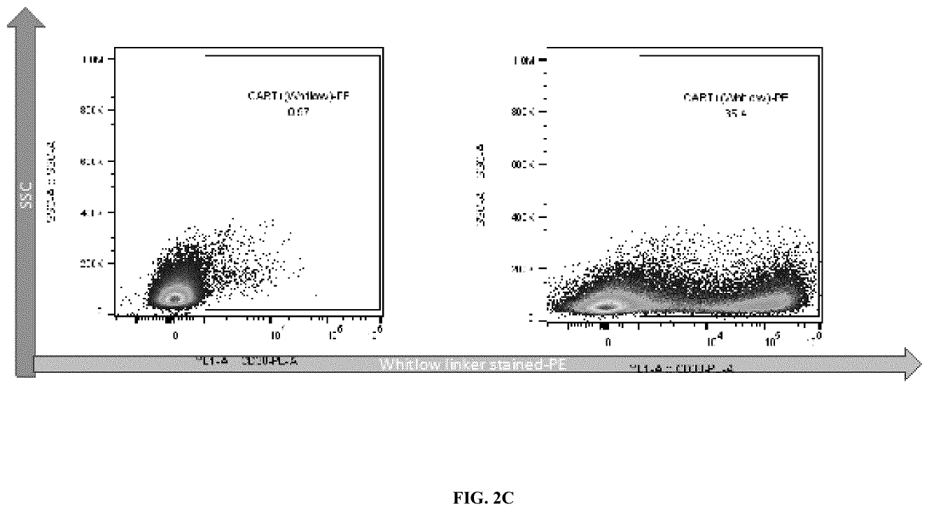

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or ±1% from the measurable value.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

The phrases “concurrent administration”, “administration in combination”, “simultaneous administration” or “administered simultaneously” as used herein, means that the compounds are administered at the same point in time or immediately following one another.

The phrase “codon optimized” as it refers to genes or coding regions of nucleic acid molecules for the transformation of various hosts, refers to the alteration of codons in the gene or coding regions of nucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA occurs.

The term “expression cassette” or “vector” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence.

The term “genetically engineered cell” or “genetically modified cell” as used herein refers to a cell modified by means of genetic engineering. The term as used herein “engineered” or “modified” thereof may refer to one or more changes of nucleic acids, such as nucleic acids within the genome of an organism. The term “engineered” or “modified” may refer to a change, addition and/or deletion of a gene. Engineered cells or modified cells can also refer to cells that contain added, deleted, and/or changed genes.

As used herein, the term “graft-versus-host” or “GVH” refers to an immune response of graft (donor) cells against host cells and tissues.

A nucleic acid sequence is “heterologous” to a second nucleic acid sequence if it originates from a foreign species, or, if from the same species, is modified by human action from its original form. For example, a heterologous promoter (or heterologous 5′ untranslated region (5′UTR)) operably linked to a coding sequence refers to a coding sequence from a species different from that from which the promoter was derived, or, if from the same species, a coding sequence which is different from naturally occurring allelic variants (for example, the 5′UTR or 3′UTR from a different gene is operably linked to a nucleic acid encoding for a co-stimulatory molecule).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level so long as the increase is statistically significant.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs, or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinas (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981). It will be appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level so long as the decrease is statistically significant.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. Administration of the therapeutic agents can be carried out at dosages and for periods of time effective for treatment of a subject.

As used herein, a “target”, “target molecule”, or “target cell” refers to a biomolecule or a cell that can be the focus of a therapeutic drug strategy, diagnostic assay, or a combination thereof, sometimes referred to as a theranostic. Therefore, a target can include, without limitation, many organic molecules that can be produced by a living organism or synthesized, for example, a protein or portion thereof, a peptide, a polysaccharide, an oligosaccharide, a sugar, a glycoprotein, a lipid, a phospholipid, a polynucleotide or portion thereof, an oligonucleotide, an aptamer, a nucleotide, a nucleoside, DNA, RNA, a DNA/RNA chimera, an antibody or fragment thereof, a receptor or a fragment thereof, a receptor ligand, a nucleic acid-protein fusion, a hapten, a nucleic acid, a virus or a portion thereof, an enzyme, a co-factor, a cytokine, a chemokine, as well as small molecules (e.g., a chemical compound), for example, primary metabolites, secondary metabolites, and other biological or chemical molecules that are capable of activating, inhibiting, or modulating a biochemical pathway or process, and/or any other affinity agent, among others.

“Therapeutically effective amount” or “therapeutically effective dose” of a composition refers to an amount that is effective to achieve a desired therapeutic result. In some embodiments, a desired therapeutic result is reduction or clearance of a pathogen. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

As used herein, the term “preventing” a disease, a disorder, or unwanted physiological event in a subject refers to the prevention of a disease, a disorder, or unwanted physiological event or prevention of a symptom of a disease, a disorder, or unwanted physiological event

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, cells, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The term “polypeptide” refers to a compound made up of a single chain of D- or L-amino acids or a mixture of D- and L-amino acids joined by peptide bonds.

The term “nucleic acid” as used herein means a polymer composed of nucleotides, e.g. deoxyribonucleotides or ribonucleotides.

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “polynucleotide” refers to a single or double stranded polymer composed of nucleotide monomers.

### Gene-Editing Systems and Engineered Cells

In some aspects, disclosed herein are compositions and methods for creating genetically modified T cells and uses thereof for treatment of cancer. The compositions and methods disclosed herein can reduce the risk of acute graft-versus-host disease.

In some aspects, disclosed herein is a genetically modified T cell comprising a nucleic acid sequence encoding a chimeric antigen receptor (CAR) polypeptide comprising a single-chain variable fragment (scFV) that specifically binds to a target molecule, wherein the nucleic acid sequence encoding the CAR is integrated into an integration site located at a miR-155 host gene.

MicroRNAs are small non-coding RNAs, with an average of 22 nucleotides in length. Most miRNAs are transcribed from DNA sequences into primary miRNAs (pri-miRNAs) and processed into precursor miRNAs (pre-miRNAs) and mature miRNAs. The term “miRNA” herein includes the primary (pri-miRNA), precursor (pre-miRNA) and/or mature forms of the miRNA. The term also includes modified forms (e.g., sequence variants) of the miRNA (e.g., 1 2, 3, 4, 5, or more nucleotides that are substituted, inserted and/or deleted). In representative embodiments, the variant substantially retains at least one biological activity of the wild-type miRNA. The term also includes variants that have been modified to resist degradation within a subject and/or within a cell. The term further includes fragments of a miRNA that substantially retain at least one biological activity of the wild-type miRNA. By “substantially retains” at least one biological activity of the wild-type miRNA means at least about 50%, 60%, 70%, 80%, 90% or more of the biological activity of the wild-type miRNA. The one or more biological activities of miRNA can include any relevant activity, including without limitation, binding activity (e.g., to a target mRNA), prevention and/or treatment of GVHD.

The term “miR-155 host gene” herein refers to DNA sequences encoding miR-155. miR 155 host gene is encoded on the + strand of human chromosome 21 from 25,562,048 to 25,575,168 (Gene: ENSG00000234883.7, Transcript: ENST00000659862.2). In some embodiments, the miR-155 host gene comprises at least 80% (e.g., at least about 80%, about 85%, about 90%, about 95%, or about 98%) identical to SEQ ID NO: 8 or a fragment thereof. In some embodiments, the integration site is at exon 1, exon 2, exon 3, or the transcriptional start site of the miR-155 host gene. In some embodiments, the integration results in deletion of mir-155 host gene or a fragment thereof. In some embodiments, the deletion of the genetically modified cell disclosed herein is in exon 1, exon 2, exon 3, or the transcriptional start site of the miR-155 host gene. In some embodiments, the deletion is in exon 3. In some embodiments, the deletion is in the transcriptional start site of the miR-155 host gene. In some embodiments, the deletion is in the promoter of the miR-155 host gene.

The genetically modified T cell can be generated by using a method comprising: obtaining a T cell;
obtaining a gene-editing system comprising a ribonucleoprotein (RNP) complex comprising a CRISPR/Cas endonuclease (Cas9) system complexed with one or more guide RNAs targeting a miR-155 host gene or a fragment thereof; and an AAV vector comprising a plasmid, nucleic acid, or construct comprising a polynucleotide sequence encoding the CAR polypeptide; wherein the polynucleotide sequence is flanked by homology arms; and
introducing the gene-editing system into the T cell; wherein the polynucleotide sequence encoding the CAR polypeptide is introduced into the T cell via infection with the AAV into the cell; wherein the RNP complex hybridizes to a target sequence within the miR-155 host gene of the T cell and the T cell's DNA repair enzymes insert the polynucleotide sequence encoding the CAR polypeptide into the miR-155 host genome of the T cell thereby creating the genetically modified T cell.

Accordingly, in some embodiments, the genetically modified T cell further comprises a ribonucleoprotein (RNP) complex comprising a CRISPR/Cas endonuclease (Cas9) system complexed with one or more guide RNAs (e.g., one, two, three, four, five or gRNAs) targeting the miR-155 host gene or a fragment thereof; and an AAV vector comprising a plasmid, nucleic acid, or construct comprising a polynucleotide sequence encoding the CAR polypeptide; wherein the polynucleotide sequence is flanked by homology arms.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. CRISPR systems are known in the art. Sec, e.g., U.S. Pat. No. 8,697,359, incorporated by reference herein in its entirety.

The terms “guide RNA”, “single guide RNA” and “synthetic guide RNA” are used interchangeably and refer to the polynucleotide sequence comprising the guide sequence, the tracr sequence and the tracr mate sequence. The term “guide sequence” refers to the about 20 bp sequence within the guide RNA that specifies the target site and may be used interchangeably with the term “guide” or “spacer”. A “crRNA” is a bacterial RNA that confers target specificity and requires tracrRNA to bind to Cas9. A “tracrRNA” is a bacterial RNA that links the crRNA to the Cas9 nuclease and typically can bind any crRNA. The sequence specificity of a Cas DNA-binding protein is determined by gRNAs, which have nucleotide base-pairing complementarity to target DNA sequences.

In some embodiments, the CRISPR/Cas9 system comprises one gRNA targeting the mir-155 host gene or a fragment thereof. In some embodiments, the CRISPR/Cas9 system comprises two gRNAs targeting the mir-155 host gene or a fragment thereof. In some embodiments, the gRNA comprises a polynucleotide sequence at least 80% (e.g., at least 80%, 85%, 90%, 95%, 98%, or 99%) identical to SEQ ID NO: 1 or 2 or a fragment thereof. In some embodiments, the CRISPR/Cas9 system comprises a first guide RNA and a second guide RNA. In some embodiments, the first guide RNA comprises a polynucleotide sequence at least 80% (e.g., at least 80%, 85%, 90%, 95%, 98%, or 99%) identical to SEQ ID NO: 1 or a fragment thereof. In some embodiments, the second guide RNA comprises a polynucleotide sequence at least 80% (e.g., at least 80%, 85%, 90%, 95%, 98%, or 99%) identical to SEQ ID NO: 2 or a fragment thereof.

The homology arms disclosed herein can target a sequence within or around a miR 155 host gene locus. In some embodiments the homology arms are 10-800 bp in length. In some embodiments, the homology arms are 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, or 800 bp in length.

In some embodiments, the homology arms can be symmetrical 30 bp homology arms, symmetrical 300 bp homology arms, symmetrical 500 bp homology arms, symmetrical 600 bp homology arms, symmetrical 800 bp homology arms, or asymmetrical 800 bp homology arms for homologous recombination (HR). In some embodiments, the homology arm comprises a sequence at least 80% (e.g., at least 80%, 85%, 90%, 95%, 98%, or 99%) identical to SEQ ID NO: 3 or 4, or a fragment thereof. In some embodiments, the AAV vector comprise a plasmid, nucleic acid, and/or construct comprising in order a left homology arm, a polynucleotide sequence encoding a chimeric antigen receptor (CAR) polypeptide, and a right homology arm. In some embodiments, the left homology arm comprises a sequence at least 80% (e.g., at least 80%, 85%, 90%, 95%, 98%, or 99%) identical to SEQ ID NO: 4. In some embodiments, the right homology arm comprises a sequence at least 80% (e.g., at least 80%, 85%, 90%, 95%, 98%, or 99%) identical to SEQ ID NO: 3.

As used herein “chimeric antigen receptor” or “CAR” refers to a chimeric receptor that targets a cancer antigen and serves to bring the cell expressing the receptor to a cancer cell expressing the target antigen. Typically, the CAR comprises a molecule that recognizes peptides derived from the tumor antigen presented by major histocompatibility (MHC) molecules, or an antibody or fragment thereof (such as, for example, a Fab′, scFv, Fv) expressed on the surface of the CAR cell that targets a cancer antigen. The target molecule of the CAR can be a cancer-related protein (including, for example, CD19, GD2, or CD33). The CAR can further comprise a transmembrane domain (for example, a CD28 transmembrane domain or a CD35 transmembrane domain) and/or a co-stimulatory domain (for example, a 2B4 domain, a CD28 co-stimulatory domain, a 4-1BB co-stimulatory domain, or any combination thereof).

The AAV vector disclosed herein can further comprise additional BHPA to minimize the effect of endogenous CD38 promoter. In some embodiments, the BHPA comprises a sequence at least 80% (e.g., at least 80%, 85%, 90%, 95%, 98%, or 99%) identical to SEQ ID NO: 7 or a fragment thereof.

In some embodiments, the AAV vector comprises a polynucleotide sequence at least 80% (e.g., at least 80%, 85%, 90%, 95%, 98%, or 99%) identical to SEQ ID NO: 6 or a fragment thereof.

In some embodiments, the T cell is a primary T cell, a T cell line, a chimeric antigen receptor (CAR)-T cell, a tumor infiltrating lymphocyte, an effector T cell, a memory T cell (e.g., CD45RA^{lo}CD45RO^{hi}CD62L^{hi} central memory T cell and CD45RA^{lo}CD45RO^{hi}CD62L^{lo} effector memory T cell), a TEMRA (terminally differentiated effector memory T cell)(CD45RA^{hi}CD45RO^{lo}CD62L^{lo}), or a stem cell-like memory T cell (CD45RA^{hi}CD45RO^{lo}CD62L^{lo}). In some embodiments, the T cell is a tumor-specific T cell. In some embodiments, the T cell is an activated T cell. In some embodiments, the T cell is a human T cell.

Also disclosed herein is a gene-editing system for engineering a CAR-T cell, said system comprising a ribonucleoprotein (RNP) complex comprising a CRISPR/Cas endonuclease (Cas9) system complexed with one or more guide RNAs targeting a miR-155 host gene or a fragment thereof; and an AAV vector comprising a plasmid, nucleic acid, or construct comprising a polynucleotide sequence encoding the CAR polypeptide; wherein the polynucleotide sequence is flanked by homology arms.

In some embodiments, the homology arm comprises a sequence at least 80% (e.g., at least 80%, 85%, 90%, 95%, 98%, or 99%) identical to SEQ ID NO: 3 or 4, or a fragment thereof. In some embodiments, the AAV vector comprise a plasmid, nucleic acid, and/or construct comprising in order a left homology arm, a polynucleotide sequence encoding a chimeric antigen receptor (CAR) polypeptide, and a right homology arm. In some embodiments, the left homology arm comprises a sequence at least 80% (e.g., at least 80%, 85%, 90%, 95%, 98%, or 99%) identical to SEQ ID NO: 4. In some embodiments, the right homology arm comprises a sequence at least 80% (e.g., at least 80%, 85%, 90%, 95%, 98%, or 99%) identical to SEQ ID NO: 3.

In some embodiments, the CRISPR/Cas9 system comprises one gRNA targeting the mir-155 host gene or a fragment thereof. In some embodiments, the CRISPR/Cas9 system comprises two gRNAs targeting the mir-155 host gene or a fragment thereof. In some embodiments, the gRNA comprises a polynucleotide sequence at least 80% (e.g., at least 80%, 85%, 90%, 95%, 98%, or 99%) identical to SEQ ID NO: 1 or 2 or a fragment thereof. In some embodiments, the CRISPR/Cas9 system comprises a first guide RNA and a second guide RNA. In some embodiments, the first guide RNA comprises a polynucleotide sequence at least 80% (e.g., at least 80%, 85%, 90%, 95%, 98%, or 99%) identical to SEQ ID NO: 1 or a fragment thereof. In some embodiments, the second guide RNA comprises a polynucleotide sequence at least 80% (e.g., at least 80%, 85%, 90%, 95%, 98%, or 99%) identical to SEQ ID NO: 2 or a fragment thereof.

In some embodiments, the AAV vector comprises a polynucleotide sequence at least 80% (e.g., at least 80%, 85%, 90%, 95%, 98%, or 99%) identical to SEQ ID NO: 6 or a fragment thereof.

### Methods of Treatment

In some aspects, disclosed herein is a method for treating cancer in a subject, comprising administering to the subject a therapeutically effective amount of the genetically modified T cell of any preceding aspect. The T cell can be cultured ex vivo for at least 2 days prior to the administration. In some embodiments, the T cell is cultured with IL-2, IL-7, or IL-15, or any combination thereof. In some embodiments, the T cell is derived from the subject or a different subject. Administration of the genetically modified T cell can prevent and/or treat acute graft-versus-host disease.

Also disclosed herein is a method of creating or manufacturing a genetically modified T cell comprising a nucleic acid sequence encoding a chimeric antigen receptor (CAR) polypeptide integrated into an integration site located at a miR-155 host gene, said method comprising
a) obtaining a T cell;
b) obtaining a gene-editing system comprising a ribonucleoprotein (RNP) complex comprising a CRISPR/Cas endonuclease (Cas9) system complexed with one or more guide RNAs targeting a miR-155 host gene or a fragment thereof; and an AAV vector comprising a plasmid, nucleic acid, or construct comprising a polynucleotide sequence encoding the CAR polypeptide; wherein the polynucleotide sequence is flanked by homology arms; and
c) introducing the gene-editing system into the T cell; wherein the polynucleotide sequence encoding the CAR polypeptide is introduced into the T cell via infection with the AAV into the cell; wherein the RNP complex hybridizes to a target sequence within the miR-155 host gene of the T cell and the T cell's DNA repair enzymes insert the polynucleotide sequence encoding the CAR polypeptide into the miR-155 host genome of the T cell thereby creating the genetically modified T cell.

The manufactured T cells can be used for treating cancer in a subject in need. Accordingly, in some aspects, disclosed herein is a method of treating cancer in a subject, said method comprising
a) obtaining a T cell; and
b) obtaining a gene-editing system comprising a ribonucleoprotein (RNP) complex comprising a CRISPR/Cas endonuclease (Cas9) system complexed with one or more guide RNAs targeting a miR-155 host gene or a fragment thereof; and an AAV vector comprising a plasmid, nucleic acid, or construct comprising a polynucleotide sequence encoding the CAR polypeptide; wherein the polynucleotide sequence is flanked by homology arms;
c) introducing the gene-editing system into the T cell; wherein the polynucleotide sequence encoding the CAR polypeptide is introduced into the T cell via infection with the AAV into the cell; wherein the RNP complex hybridizes to a target sequence within the miR-155 host gene of the T cell and the T cell's DNA repair enzymes insert the polynucleotide sequence encoding the CAR polypeptide into the miR-155 host genome of the T cell thereby creating the genetically modified T cell; and
d) administering to the subject a therapeutically effective amount of the genetically modified T cell.

In some embodiments, the T cell is a primary T cell, a T cell line, a chimeric antigen receptor (CAR)-T cell, a tumor infiltrating lymphocyte, an effector T cell, a memory T cell (e.g., CD45RA^{lo}CD45RO^{hi}CD62L^{hi} central memory T cell and CD45RA^{lo}CD45RO^{hi}CD62L^{lo} effector memory T cell), a TEMRA (terminally differentiated effector memory T cell)(CD45RA^{hi}CD45RO^{lo}CD62L^{lo}), or a stem cell-like memory T cell (CD45RA^{hi}CD45RO^{lo}CD62L^{lo}). In some embodiments, the T cell is a tumor-specific T cell. In some embodiments, the T cell is an activated T cell. In some embodiments, the T cell is a human T cell. The T cell can be derived from the subject or a different subject.

In some embodiments, the T cell is cultured ex vivo for at least 2 days prior to the administration (e.g., the T cell is cultured ex vivo for at least 2 days, 3 days, 4 days, 5 days, 6 days, 7 days, 8 days, 9 days, or 10 days prior to the administration). In some embodiments, the T cell is culture ex vivo for at least 7 days prior to the administration. T cells can be cultured with, including, for example, IL-2, anti-CD3 antibody, and/or anti-CD28 antibody. In some embodiments, the T cell is derived from the subject.

In some embodiments, the integration site is at exon 1, exon 2, exon 3, or the transcriptional start site of the miR-155 host gene. In some embodiments, the integration results in deletion of mir-155 host gene or a fragment thereof. In some embodiments, the deletion of the genetically modified cell disclosed herein is in exon 1, exon 2, exon 3, or the transcriptional start site of the miR-155 host gene.

In some embodiments, the homology arm comprises a sequence at least 80% (e.g., at least 80%, 85%, 90%, 95%, 98%, or 99%) identical to SEQ ID NO: 3 or 4, or a fragment thereof. In some embodiments, the AAV vector comprise a plasmid, nucleic acid, and/or construct comprising in order a left homology arm, a polynucleotide sequence encoding a chimeric antigen receptor (CAR) polypeptide, and a right homology arm. In some embodiments, the left homology arm comprises a sequence at least 80% (e.g., at least 80%, 85%, 90%, 95%, 98%, or 99%) identical to SEQ ID NO: 4. In some embodiments, the right homology arm comprises a sequence at least 80% (e.g., at least 80%, 85%, 90%, 95%, 98%, or 99%) identical to SEQ ID NO: 3.

In some embodiments, the CRISPR/Cas9 system comprises one gRNA targeting the mir-155 host gene or a fragment thereof. In some embodiments, the CRISPR/Cas9 system comprises two gRNAs targeting the mir-155 host gene or a fragment thereof. In some embodiments, the gRNA comprises a polynucleotide sequence at least 80% (e.g., at least 80%, 85%, 90%, 95%, 98%, or 99%) identical to SEQ ID NO: 1 or 2 or a fragment thereof. In some embodiments, the CRISPR/Cas9 system comprises a first guide RNA and a second guide RNA. In some embodiments, the first guide RNA comprises a polynucleotide sequence at least 80% (e.g., at least 80%, 85%, 90%, 95%, 98%, or 99%) identical to SEQ ID NO: 1 or a fragment thereof. In some embodiments, the second guide RNA comprises a polynucleotide sequence at least 80% (e.g., at least 80%, 85%, 90%, 95%, 98%, or 99%) identical to SEQ ID NO: 2 or a fragment thereof.

In some embodiments, the AAV vector comprises a polynucleotide sequence at least 80% (e.g., at least 80%, 85%, 90%, 95%, 98%, or 99%) identical to SEQ ID NO: 6 or a fragment thereof.

Exemplary cancers include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendocrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some examples, the subject is diagnosed as having a blood cancer (including, for example, Acute leukemia, Chronic leukemia, Hodgkin's lymphoma, Multiple myeloma, Neuroblastoma, or Non-Hodgkin's lymphoma). It should be understood and herein contemplated that the cells and methods disclosed herein are effective in treating a blood cancer in a subject without causing acute graft-versus-host disease.

It should be understood and herein contemplated that the compositions and methods described herein can be used for prevention and/or treatment of aGVHD in the subject.

As used herein, the term “graft-versus-host disease” or “GVHD” refers to a condition, including acute and chronic, resulting from transplanted (graft) cell effects on host cells and tissues resulting from an allogeneic hematopoietic cell transplant. In other words, donor immune cells infused within the graft or donor immune cells that develop from the stem cells, may see the patient's (host) cells as foreign and turn against them with an immune response. As examples, patients who have had a blood or marrow transplant from someone else are at risk of having acute GVHD. Even donors who are HLA-matched with the recipient can cause GVHD because the donor cells can potentially also make an immune response against minor antigen differences in the recipient. Acute graft-versus-host disease (GVHD) is a disorder caused by donor immune cells in patients who have had an allogeneic marrow or blood cell transplantation. The most commonly affected tissues are skin, intestine and liver. In severe cases, GVHD can cause blistering in the skin or excessive diarrhea and wasting. Also, inflammation caused by donor immune cells in the liver can cause obstruction that causes jaundice. Other tissues such as lung and thymus may also become affected. The diagnosis is usually confirmed by looking at a small piece of skin, liver, stomach or intestine with a microscope for observation of specific inflammatory characteristics. The symptoms of acute GVHD further comprises an increase of white blood cell counts and proinflammatory cytokine levels. The symptoms of acute GVHD usually begins within the first 3 months after the transplant. In some cases, it can persist, come back or begin more than 3 months after the transplant. In some examples, the compositions and methods disclosed herein are used for treatment and/or prevention of acute GVHD.

The compositions and methods disclosed herein for preventing and/or treating acute GVHD can be a prevention and/or treatment of one or more of blistering in the skin, skin rashes, abdominal cramps, excessive diarrhea, inflammation in the liver, intestine, lung, thymus, jaundice, and/or nausea.

Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, intravaginal, by inhalation, via an implanted reservoir, or via a transdermal patch, and the like.

Dosing frequency for the genetically modified cell disclosed herein, includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiments, dosing frequency for the genetically modified cell disclosed herein includes at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks or once a week. In some embodiments, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiments, the dosing frequency for genetically modified cell includes, but is not limited to, at least once a day, twice a day, or three times a day. In some embodiments, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiments, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiments, the interval between each administration is less than about 4 months, less than about 3 months, less than about 2 months, less than about a month, less than about 3 weeks, less than about 2 weeks, or less than less than about a week. In some embodiments, the interval between each administration is constant. For example, the administration can be carried out daily, every two days, every three days, every four days, every five days, or weekly. In some examples, the administration can be carried out every week, every two weeks, or every two months.

The dose of T cells can vary from, for example, from about 1×10⁵ to about 200×10⁶ CD3+ T cells/kg body weight. In some embodiments, the dose of T cells is from about 1×10⁶ to about 200×10⁶ CD3+ T cells/kg body weight. In some embodiments, the dose of T cells is about 1×10⁶ CD3+ T cells/kg body weight, about 2×10⁶ CD3+ T cells/kg body weight, about 3×10⁶ CD3+ T cells/kg body weight, about 5×10⁶ CD3+ T cells/kg body weight, about 10×10⁶ CD3+ T cells/kg body weight, about 20×10⁶ CD3+ T cells/kg body weight, about 50×10⁶ CD3+ T cells/kg body weight, about 80×10⁶ CD3+ T cells/kg body weight, about 100×10⁶ CD3+ T cells/kg body weight, about 150×10⁶ CD3+ T cells/kg body weight, or about 200×10⁶ CD3+ T cells/kg body weight.

### EXAMPLES

The following examples are set forth below to illustrate the compositions, cells, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. miR155 as the Integration Site to Overcome Acute Graft Versus Host Disease (GVHD) of Gene-Modified Immune Effector Cells

Acute graft versus host disease is a common complication that occurs after allogeneic hematopoietic stem cell transplantation. MIR155 is progressively expressed in many cancers that makes suppression of this gene is therapeutically relevant. Previous studies showed that miR155 is over-expressed in donors with GVHD T cells. It has been shown Graft versus host disease can be prevented by knocking out miR155 in donor T cells.

Previous studies developed approaches to generate CAR-NK cells using Cas9/RNP+AAV6 to site direct DNA encoding CARs in AAVS1 and CD38 loci in NK cells and T cells. The present example provides novel constructs for gene knock-in at the MIR155 locus allowing simultaneous MIR155 knock-out and CAR knock-in in T cells. For a complete deletion, double gRNA strategy was used to remove the MIR155 locus from the genome.

To generate these novel cells, this study developed homology arms for integration of any transgene in the MIR155 locus for simultaneous MIR155 KO and CAR insertion. Based on successful miR155 targeting studies, homology arms were designed for site directed gene insertion:

Right Homology Arm miR155: (SEQ ID NO: 3)

GGGGACATTTTCAGATAGATTCGTTTGTAGGCAAACCTCCATTGCTTGT
ATCACATTTCCTGAAAGAATAAAGGTAAAACTTCAACTATGTATTACAG
AAAGAAAAATTCAGCCTGAACCCTACCCTTATAAAACAGGTTAATTGGG
TTTTAATTTTCATAAATCATAAAGGACTATTTTGAACATTTGGGCCTTT
AATTGTCTAGCTCCTAGATGAAGTACAAATCAGaaaaaaaaaaaaCTGT
ACTGTGTCAGAATGCAAGCTTTCCTCTTTGCATTTTGGCATTTGAAAAC
TCCGAAGAGCGGTTTTTGTTTTTTATTTAAAGAAGATGATACATATGTG
TACCCGATTCAAAACTAGAGAATAGAATTTAAAACATAATTTTCAAAGT
CTTCAAATATGCCTAAAGGTAACAATGTCATCTTTTAATTGCCAATTTC
TCTACCACTTTCAAAAAATTACTTCCAAGGATTTAATGAGCTCCTTCCT
TTCAACAGAAAATGGACTATTTTCCTTTCAGATTTACTATATGCTGTCA
CTCCAGCTTTATAACCGCATGTGCATACACAAACATTTCTTTCTCTCTT
GCAGGTGGCACA

Left Homology Arm miR155: (SEQ ID NO: 4)

CCGTGGGAGGATGACAAAGAAGCATGAGTCACCCTGCTGGATAAACTTA
GACTTCAGGCTTTATCATTTTTCAATCTGTTAATCATAATCTGGTCACT
GGGATGTTCAACCTTAAACTAAGTTTTGAAAGTAAGGTTATTTAAAAGA
TTTATCAGTAGTATCCTAAATGCAAACATTTTCATTTAAATGTCAAGCC
CATGTTTGTTTTTATCATTAACAGAAAATATATTCATGTCATTCTTAAT
TGCAGGTTTTGGCTTGTTCATTATAATGTTCATAAACACCTTTGATTCA
ACTGTTAGAAATGTGGGCTAAACACAAATTTCTATAATATTTTTGTAGT
TAAAAATTAGAAGGACTACTAACCTCCAGTTATATCATGGATTGTCTGG
CAACGTTTTTTAAAAGATTTAGAAACTGGTACTTTCCCCCAGGTAACGA
TTTTCTGTTCAGGCAACTTCAGTTTAAAATTAATACTTTTATTTGACTC
TTAAAGGGAAACTGAAAGGCTATGAAGCTGAATTTTTTTAATGAAATAT
TTTTAACAGTTAGCAGGGTAAATAACATCTGACAGCTAATGAGATATTT
TTTCCATACAAG

This study has designed the regulatory elements for highly efficient simultaneous gene KO and CAR expression. This includes an additional BHPA to minimize the effect of endogenous CD38 promoter. (SEQ ID NO: 7)

CCTCGACTGTGCCTTCTAGTTGCCAGCCATCTGTTGTTTGCCCCTCCCC
CGTGCCTTCCTTGACCCTGGAAGGTGCCACTCCCACTGTCCTTTCCTAA
TAAAATGAGGAAATTGCATCGCATTGTCTGAGTAGGTGTCATTCTATTC

This study designed a universal construct for cloning the DNA-encoding CAR in AAV vectors for simultaneous MIR155 KO and a transgene expression. The construct now can be used for any CAR or any gene integration and transgene expression in T cells.

gRNA Sequences Used to Target Mir155:

gRNA1:
(SEQ ID NO: 1)
TCTTGCAGGTGGCACAAACC
gRNA2:
(SEQ ID NO: 2)
ATGGAACAAATTGCTGCCGT

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### SEQUENCES

gRNA Sequences Used to Target Mir155:

gRNA1:
(SEQ ID NO: 1)
TCTTGCAGGTGGCACAAACC
gRNA2:
(SEQ ID NO: 2)
ATGGAACAAATTGCTGCCGT

### Right Homology Arm miR 155 (SEQ ID NO: 3)

GGGGACATTTTCAGATAGATTCGTTTGTAGGCAAACCTCCATTGCTTGT
ATCACATTTCCTGAAAGAATAAAGGTAAAACTTCAACTATGTATTACAG
AAAGAAAAATTCAGCCTGAACCCTACCCTTATAAAACAGGTTAATTGGG
TTTTAATTTTCATAAATCATAAAGGACTATTTTGAACATTTGGGCCTTT
AATTGTCTAGCTCCTAGATGAAGTACAAATCAGaaaaaaaaaaaaCTGT
ACTGTGTCAGAATGCAAGCTTTCCTCTTTGCATTTTGGCATTTGAAAAC
TCCGAAGAGCGGTTTTTGTTTTTTATTTAAAGAAGATGATACATATGTG
TACCCGATTCAAAACTAGAGAATAGAATTTAAAACATAATTTTCAAAGT
CTTCAAATATGCCTAAAGGTAACAATGTCATCTTTTAATTGCCAATTTC
TCTACCACTTTCAAAAAATTACTTCCAAGGATTTAATGAGCTCCTTCCT
TTCAACAGAAAATGGACTATTTTCCTTTCAGATTTACTATATGCTGTCA
CTCCAGCTTTATAACCGCATGTGCATACACAAACATTTCTTTCTCTCTT
GCAGGTGGCACA

### Left Homology Arm miR 155 (SEQ ID NO: 4)

CCGTGGGAGGATGACAAAGAAGCATGAGTCACCCTGCTGGATAAACTTA
GACTTCAGGCTTTATCATTTTTCAATCTGTTAATCATAATCTGGTCACT
GGGATGTTCAACCTTAAACTAAGTTTTGAAAGTAAGGTTATTTAAAAGA
TTTATCAGTAGTATCCTAAATGCAAACATTTTCATTTAAATGTCAAGCC
CATGTTTGTTTTTATCATTAACAGAAAATATATTCATGTCATTCTTAAT
TGCAGGTTTTGGCTTGTTCATTATAATGTTCATAAACACCTTTGATTCA
ACTGTTAGAAATGTGGGCTAAACACAAATTTCTATAATATTTTTGTAGT
TAAAAATTAGAAGGACTACTAACCTCCAGTTATATCATGGATTGTCTGG
CAACGTTTTTTAAAAGATTTAGAAACTGGTACTTTCCCCCAGGTAACGA
TTTTCTGTTCAGGCAACTTCAGTTTAAAATTAATACTTTTATTTGACTC
TTAAAGGGAAACTGAAAGGCTATGAAGCTGAATTTTTTTAATGAAATAT
TTTTAACAGTTAGCAGGGTAAATAACATCTGACAGCTAATGAGATATTT
TTTCCATACAAG

### BHPA Sequence (SEQ ID NO: 5)

CCTCGACTGTGCCTTCTAGTTGCCAGCCATCTGTTGTTTGCCCCTCCCC
CGTGCCTTCCTTGACCCTGGAAGGTGCCACTCCCACTGTCCTTTCCTAA
TAAAATGAGGAAATTGCATCGCATTGTCTGAGTAGGTGTCATTCTATTC

Sequence of a CD33 CAR integrated in the miR 155 locus using Cas9/RNP+AAV approach map. Full length of a CAR (MIR155KO-CD33CAR-v4). (SEQ ID NO: 6)

CCTGCAGGCAGCTGCGCGCTCGCTCGCTCACTGAGGCCGCCCGGGCGTCGGGCG
ACCTTTGGTCGCCCGGCCTCAGTGAGCGAGCGAGCGCGCAGAGAGGGAGTGGCC
AACTCCATCACTAGGGGTTCCTGCGGCCGGCGCGCCGGGGACATTTTCAGATAG
ATTCGTTTGTAGGCAAACCTCCATTGCTTGTATCACATTTCCTGAAAGAATAAAG
GTAAAACTTCAACTATGTATTACAGAAAGAAAAATTCAGCCTGAACCCTACCCTT
ATAAAACAGGTTAATTGGGTTTTAATTTTCATAAATCATAAAGGACTATTTTGAA
CATTTGGGCCTTTAATTGTCTAGCTCCTAGATGAAGTACAAATCAGaaaaaaaaaaaaC
TGTACTGTGTCAGAATGCAAGCTTTCCTCTTTGCATTTTGGCATTTGAAAACTCCG
AAGAGCGGTTTTTGTTTTTTATTTAAAGAAGATGATACATATGTGTACCCGATTC
AAAACTAGAGAATAGAATTTAAAACATAATTTTCAAAGTCTTCAAATATGCCTAA
AGGTAACAATGTCATCTTTTAATTGCCAATTTCTCTACCACTTTCAAAAAATTACT
TCCAAGGATTTAATGAGCTCCTTCCTTTCAACAGAAAATGGACTATTTTCCTTTCA
GATTTACTATATGCTGTCACTCCAGCTTTATAACCGCATGTGCATACACAAACAT
TTCTTTCTCTCTTGCAGGTGGCACACCTCGACTGTGCCTTCTAGTTGCCAGCCATC
TGTTGTTTGCCCCTCCCCCGTGCCTTCCTTGACCCTGGAAGGTGCCACTCCCACTG
TCCTTTCCTAATAAAATGAGGAAATTGCATCGCATTGTCTGAGTAGGTGTCATTC
TATTCATCGATCACGAGACTAGCCTCGAGAAGCTTGATATCGAATTCCACGGGGT
TGGACGCGTCTTAATTAAGGATCCAAGGTCAGGAACAGAGAAACAGGAGAATAT
GGGCCAAACAGGATATCTGTGGTAAGCAGTTCCTGCCCCGGCTCAGGGCCAAGA
ACAGTTGGAACAGCAGAATATGGGCCAAACAGGATATCTGTGGTAAGCAGTTCC
TGCCCCGGCTCAGGGCCAAGAACAGATGGTCCCCAGATGCGGTCCCGCCCTCAG
CAGTTTCTAGAGAACCATCAGATGTTTCCAGGGTGCCCCAAGGACCTGAAATGA
CCCTGTGCCTTATTTGAACTAACCAATCAGTTCGCTTCTCGCTTCTGTTCGCGCGC
TTCTGCTCCCCGAGCTCTATATAAGCAGAGCTCGTTTAGTGAACCGTCAGATCGC
CTGGAGACGCCATCCACGCTGTTTTGACCTCCATAGAAGACACCGACTCTAGAGG
ATCGATCCCCCGGGCTGCAGGAATTCAAGCGAGAAGACAAGGGCAGAAAGCAC
CGCCACCATGCTGCTGCTGGTGACCTCTCTGCTGCTGTGCGAGCTGCCACACCCA
GCCTTCCTGCTGATCCCAGACATCCAGATGACACAGAGCCCCAGCTCCCTGAGCG
CCTCCGTGGGCGACAGAGTGACCATCACATGTAGGGCCTCTGAGAGCGTGGATA
ACTATGGCATCAGCTTCATGAATTGGTTTCAGCAGAAGCCTGGCGGCGCCCCAAA
GCTGCTGATCTACGCAGCCAGCATGCAGGGCTCCGGCGTGCCCTCTCGGTTCTCC
GGCTCTGGCAGCGGCACCGACTTCACCCTGACAATCTCTAGCCTGCAGCCAGACG
ATTTCGCCACATACTATTGCCAGCAGAGCAAGGAGGTGCCCTGGACCTTTGGCCA
GGGCACAAAGGTGGAGATCAAGGGCTCCACCTCTGGCAGCGGCAAGCCTGGCAG
CGGAGAGGGCTCCACAAAGGGACAGGTGCAGCTGGTGCAGTCCGGAGCCGAGG
TGAAGAAGCCAGGCTCCTCTGTGAAGGTGTCTTGTAAGGCCAGCGGCTATACCTT
CACAGACTACAACATGCACTGGGTGCGCCAGGCACCAGGACAGGGCCTGGAGTG
GATCGGCTACATCTATCCTTACAACGGCGGCACCGGCTATAATCAGAAGTTTAAG
TCCAAGGCCACCATCACAGCCGATGAGTCTACCAATACAGCCTACATGGAGCTG
AGCAGCCTGCGGTCCGAGGACACAGCCGTGTACTATTGCGCCCGGGGCAGACCC
GCTATGGACTATTGGGGCCAGGGCACCCTGGTGACAGTGTCTAGCACAACAACT
CCAGCACCCCGCCCACCTACTCCCGCTCCTACAATCGCCTCTCAGCCCCTGTCCCT
GCGCCCAGAAGCCTGTCGCCCCGCAGCAGGAGGAGCAGTGCACACCAGGGGCCT
GGACTTCGCCTGCGATATCTACATCTGGGCTCCTCTGGCTGGAACTTGTGGCGTG
CTGCTGCTGTCACTGGTCATTACCCTGTATTGTAAGCGGGGCAGAAAGAAGCTGC
TGTACATCTTCAAGCAGCCCTTTATGAGGCCTGTGCAGACCACACAGGAGGAGG
ACGGCTGCAGCTGTCGGTTCCCCGAGGAGGAGGAGGGCGGCTGCGAGCTGCGGG
TGAAGTTTAGCAGATCCGCCGATGCACCAGCATATCAGCAGGGACAGAATCAGC
TGTACAACGAGCTGAATCTGGGCAGGCGCGAGGAGTACGACGTGCTGGATAAGA
GGCGGGGCCGGGACCCCGAGATGGGAGGCAAGCCCAGGCGCAAGAACCCTCAG
GAGGGCCTGTATAATGAGCTGCAGAAGGACAAGATGGCCGAGGCCTACAGCGA
GATCGGCATGAAGGGAGAGCGGAGAAGGGGCAAGGGACACGATGGCCTGTATC
AGGGCCTGTCCACCGCCACAAAGGACACCTACGATGCACTGCACATGCAGGCCC
TGCCACCTCGGTGAAAGTAACGCCCTCGACTGTGCCTTCTAGTTGCCAGCCATCT
GTTGTTTGCCCCTCCCCCGTGCCTTCCTTGACCCTGGAAGGTGCCACTCCCACTGT
CCTTTCCTAATAAAATGAGGAAATTGCATCGCATTGTCTGAGTAGGTGTCATTCT
ATTCCCGTGGGAGGATGACAAAGAAGCATGAGTCACCCTGCTGGATAAACTTAG
ACTTCAGGCTTTATCATTTTTCAATCTGTTAATCATAATCTGGTCACTGGGATGTT
CAACCTTAAACTAAGTTTTGAAAGTAAGGTTATTTAAAAGATTTATCAGTAGTAT
CCTAAATGCAAACATTTTCATTTAAATGTCAAGCCCATGTTTGTTTTTATCATTAA
CAGAAAATATATTCATGTCATTCTTAATTGCAGGTTTTGGCTTGTTCATTATAATG
TTCATAAACACCTTTGATTCAACTGTTAGAAATGTGGGCTAAACACAAATTTCTA
TAATATTTTTGTAGTTAAAAATTAGAAGGACTACTAACCTCCAGTTATATCATGG
ATTGTCTGGCAACGTTTTTTAAAAGATTTAGAAACTGGTACTTTCCCCCAGGTAA
CGATTTTCTGTTCAGGCAACTTCAGTTTAAAATTAATACTTTTATTTGACTCTTAA
AGGGAAACTGAAAGGCTATGAAGCTGAATTTTTTTAATGAAATATTTTTAACAGT
TAGCAGGGTAAATAACATCTGACAGCTAATGAGATATTTTTTCCATACAAGGCGG
CCGCAGGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGGGCGG
CCTCAGTGAGCGAGCGAGCGCGCAGCTGCCTGCAGGGGCGCCTGATGCGGTATT
TTCTCCTTACGCATCTGTGCGGTATTTCACACCGCATACGTCAAAGCAACCATAG
TACGCGCCCTGTAGCGGCGCATTAAGCGCGGCGGGTGTGGTGGTTACGCGCAGC
GTGACCGCTACACTTGCCAGCGCCTTAGCGCCCGCTCCTTTCGCTTTCTTCCCTTC
CTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTTGGG
TGATGGTTCACGTAGTGGGCCATCGCCCTGATAGACGGTTTTTCGCCCTTTGACG
TTGGAGTCCACGTTCTTTAATAGTGGACTCTTGTTCCAAACTGGAACAACACTCA
ACTCTATCTCGGGCTATTCTTTTGATTTATAAGGGATTTTGCCGATTTCGGTCTAT
TGGTTAAAAAATGAGCTGATTTAACAAAAATTTAACGCGAATTTTAACAAAATAT
TAACGTTTACAATTTTATGGTGCACTCTCAGTACAATCTGCTCTGATGCCGCATA
GTTAAGCCAGCCCCGACACCCGCCAACACCCGCTGACGCGCCCTGACGGGCTTG
TCTGCTCCCGGCATCCGCTTACAGACAAGCTGTGACCGTCTCCGGGAGCTGCATG
TGTCAGAGGTTTTCACCGTCATCACCGAAACGCGCGAGACGAAAGGGCCTCGTG
ATACGCCTATTTTTATAGGTTAATGTCATGATAATAATGGTTTCTTAGACGTCAGG
TGGCACTTTTCGGGGAAATGTGCGCGGAACCCCTATTTGTTTATTTTTCTAAATAC
ATTCAAATATGTATCCGCTCATGAGACAATAACCCTGATAAATGCTTCAATAATA
TTGAAAAAGGAAGAGTatgattgaacaagatggattgcacgcaggttctccggccgcttgggtggagaggctattcgg
ctatgactgggcacaacagacaatcggctgctctgatgccgccgtgttccggctgtcagcgcaggggcgcccggttctttttgtca
agaccgacctgtccggtgccctgaatgaactgcaggacgaggcagcgcggctatcgtggctggccacgacgggcgttccttgcgca
gctgtgctcgacgttgtcactgaagcgggaagggactggctgctattgggcgaagtgccggggcaggatctcctgtcatctcacct
tgctcctgccgagaaagtatccatcatggctgatgcaatgcggcggctgcatacgcttgatccggctacctgcccattcgaccacc
aagcgaaacatcgcatcgagcgagcacgtactcggatggaagccggtcttgtcgatcaggatgatctggacgaagagcatcagggg
ctcgcgccagccgaactgttcgccaggctcaaggcgcgcatgcccgacggcgaggatctcgtcgtgacccatggcgatgcctgctt
gccgaatatcatggtggaaaatggccgcttttctggattcatcgactgtggccggctgggtgtggcggaccgctatcaggacatag
cgttggctacccgtgatattgctgaagagcttggcggcgaatgggctgaccgcttcctcgtgctttacggtatcgccgctcccgat
tcgcagcgcatcgccttctatcgccttcttgacgagttcttctgaCTGTCAGACCAAGTTTACTCATATATACTTTAGATTG
ATTTAAAACTTCATTTTTAATTTAAAAGGATCTAGGTGAAGATCCTTTTTGATAAT
CTCATGACCAAAATCCCTTAACGTGAGTTTTCGTTCCACTGAGCGTCAGACCCCG
TAGAAAAGATCAAAGGATCTTCTTGAGATCCTTTTTTTCTGCGCGTAATCTGCTGC
TTGCAAACAAAAAAACCACCGCTACCAGCGGTGGTTTGTTTGCCGGATCAAGAG
CTACCAACTCTTTTTCCGAAGGTAACTGGCTTCAGCAGAGCGCAGATACCAAATA
CTGTTCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGGCGAT
AAGTCGTGTCTTACCGGGTTGGACTCAAGACGATAGTTACCGGATAAGGCGCAG
CGGTCGGGCTGAACGGGGGGTTCGTGCACACAGCCCAGCTTGGAGCGAACGACC
TACACCGAACTGAGATACCTACAGCGTGAGCTATGAGAAAGCGCCACGCTTCCC
GAAGGGAGAAAGGCGGACAGGTATCCGGTAAGCGGCAGGGTCGGAACAGGAGA
GCGCACGAGGGAGCTTCCAGGGGGAAACGCCTGGTATCTTTATAGTCCTGTCGG
GTTTCGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTTTTGCT
GGCCTTTTGCTCACATGT

### BHPA (SEQ ID NO: 7)

CCTCGACTGTGCCTTCTAGTTGCCAGCCATCTGTTGTTTGCCCCTCCCC
CGTGCCTTCCTTGACCCTGGAAGGTGCCACTCCCACTGTCCTTTCCTAA
TAAAATGAGGAAATTGCATCGCATTGTCTGAGTAGGTGTCATTCTATTC

>MIR 155HG with PROMOTER
(SEQ ID NO: 8)

MIR155HG is encoded on the + strand of human chromosome 21 from 25.562.048 to 25.575.168 (Gene: ENSG00000234883.7. Transcript: ENST00000659862.2). Promoter and introns are in lowercase, exons are in uppercase

gatcaagatgtcagcaggactggttcctcctgagagctgtgagggaaggatctgtttcaagcccctctccttggctggtagatggccat
cttttcccaatgtttcttcacactgtcttccttctatatgtgtttctttctctacacatcatgttctttttctaagaacaccagtcatattggatt
aggagccaaccctattttaataagacctcatcttgactaattacatccacaaaggccttgtttccacataaggttatattctgaagtactggttggt
ctacagccataccaccccacacacacccgatctcatctgaagtactgggtgttaggacatcaatatatgaatttgggaggtgggggga
gggggacacaattcagcccacaacaacatccatggggtacttgtcccggcaggtggccagctcttactttctcaagctatccatttgga
gcaggtccttttttttttttctttttttttttttttgagacagtctcgctcgcattccaggctggaatgcagtggcgcgatcttggctcaccgcaaa
ctccgcctcccgcattcatgccattctcctgcctcagcctccagagtagctgggactacaggtgcccgccatcatgcccggctaatttttt
tttcttttttgtatttttagtagattcagggtttcaccattctagccaggatggtctcaatctcctgacctcatgatccagtcacctcagcctccc
aaagtgctgggattacaggatgagccaccgcgcctggcctggagcaggtactttttatccatcaccttatttaatcatcatatccctcttag
tctgctagggttgccataagaaaataccatagactggatggctgatacaacaacaatttattttctcacattctggaggctagaaagttcca
gatcaaggtcctgcagggttcggtttctggtgaggactctcttcctggcttacaggaaaccaccatcccattgtgtgctcacatgacctct
gctttgactgatttctgagagaaagggaattctggtgtctcttcctcttctcataagagcactagctctatgggattagtgatccacccttttg
acctcatttaacctttatcacctcctcacaggccctatctccaaatggggataacattggggatcagggcttcaacatataaagttgaaga
gaacatgattcagtctatagtaatccctgatgggagacatcattattgtcatttacaggaagctgagacataaaaagtttaacgttcagatt
ccacagctagaattagtgtaaaatgcaggtgtatttgactcctaaaacccatttatccatccactattctgggtgtctcaaactgggaccta
aaatgcggaggtttgcacatgacctgaaattacttccaccactgggagaagggaggattgagcaatttcctgtttctattaaagtgcattat
acaaacttgtcattctggggatgaaaggtcaccctagaattgcctatgggcaatttcttatagttcaacctagaatgagaaatgggaaatt
cagaaaggcattgtaggcatctgtaaccagcagagggacgtgccccacctgggggggaccatgcatccttgccacatgccccactg
cacaacttccccagctcctcaacgtcacatggatctggaaagcagggagactggactacggagccagccctccagggttggaactga
gtttgaatctcagcttgactacttactaggatctaaggtaacatgtttaatctctctgtgctcagtttcctcatctggaaatagagattataatg
ctcctccctcatatgatcatagtgaggactgaaagagttaatgcatataaggagtgttaggacaggatctggcacatggtaaatgctttat
aagtgttagttgttatcatcatgattattcttgtttgaattataagagaaaaagcatgtatcttaagaatagagggttttaaaatgctcccaagt
tccttaaccaacctgagccatctgtaaattaagtactatgggatttccagctctgacatgtattctgacatgtaactggcatcagtttaataca
aactattctaaaatgtcttgtgcccttgacagggaggtccaagtactggatacttgcaatgcaatccagcagtggccccgtctttcttacaa
aaaggccccagtcacatgttgatgaggctagatctattcctgtcctccttctcttccccatatttccttattcttcttcaagtctaaacgtttatt
gaactgagacccatgaatgagttactcgactaggcttgtaggataaacttgccaggctttagatctatttgttcctttccatcccccaaaatc
aaatggctgctccaggaaaatgttccccttgtggcagggtccgggagaaaagagagaagcgacaaaaccaaaaattaaaacgaccg
aagtcccatatgctccaggaatatgtcctggagatgggagtggagggcagggggagaatgttgttgaggtcaaaatttttgaagttttaa
gtcctatatcttgacatcccgagtataaatgcgggtaccagacacagtacaaacgttctcaaagcccagttacgtattccaaaccaaacg
cgggctcttgaagggtgatgaggtagggatgaaatccaggatcgcctgaagaccatttcttcctctcttagggacctgctggtctccag
ctgattcggtccaggaggaaaaacctcccacttgctcctctcgggctccctgcaaggagagagtagagacactcctgccacccagttg
caagaagtcgccacttccccctccagccgactgaaagttcgggcgacgtctgggccgtcatttgaaggcgtttccttttctttaagaaca
aaggttggagcccaagccttgcggcgcggtgcaggaaagtacacggcgtgtgttgagagaaaaaaaaTACACACACGC
AATGACCCACGAGAAAGGGAAAGGGGAAAACACCAACTACCCGGGCGCTGGGC
TTTTTCGACTTTTCCTTTAAAAAGAAAAAAGTTTTTCAAGCTgtaggttccaagaacaggcag
gaggggggagaagggggggggggttgcagaaaaggcgcctggtcggttatgagtcacaagtgagttataaaagggtcgcacgttc
gcagGCGCGGGCTTCCTGTGCGCGGCCGAGCCCGGGCCCAGCGCCGCCTGCAGCCT
CGGGAAGGGAGCGGATAGCGGAGCCCCGAGCCGCCCGCAGAGCAAGCGCGGGG
AACCAAGGAGACGCTCCTGGCACTGCAGgtacgccgacttcagtctcgcgctcccgcccgcctttcctctct
tgaacgtggcagggacgccgggggacttcggtgcgagggtcaccgccgggttaactggcgaggcaaggcgggggcagcgcgca
cgtggccgtggagcccggcctggtcccgcgcgcgcctgcgggtgccccctggggactcagtggtgtcgcctcgcccgggaccag
agattgcgctggatggattcccgcgggcagaggcagggggaaggaggggtgttcgaaacctaatacttgagcttctttgcaaagtttc
cttggatggttggggacgtacctgtataatggccctggaccagcttccctgttggagtggccagagaagtgtgtaaaacacactagag
gggcagggtggaaaaagagactgccttcaaaacttgtatcttttcgatttcattttgaaaaataactacaaatctattttaattttacaaagtta
gactcatagcattttagatatcaatgtcttcatttaacagaagtgaagatggagcaaacgctcaatcagcgtctgtatttattcgctcctgttg
tgccagggtgcgtttttgccgagcggttgcctttctttactcacaaaacccccttgatgtctgtcctccacgttttacgagggagagccgg
atcttttgaagtttgtatcatctaaagcaggtatattgggatgactatggatagaatttaacctgaaaacactgaagttgacagctgacaaa
gatataagaatccaaagtatgttaaaaattaaggagctgaggcccacagaagtgaagttacttttccagcatcacacagcagatctagg
accctgtgtgatttgaaaaggccaaggtatctaatgatgctcataatttagtttacattttgtgtcaaatagatgaggtcaagtggacattatg
tgtatattccaaagccaaaattactgcttgtaatatccattcatctctccgaagacgtagtgcctcttttatatggctttaacctaatgtataatc
gaagtgaattagggcagccagacttttaatcacaggactgatcttttaaaaacgggtatatgtaaagttaattgcttatctatgtgaacagta
atatacactataaaaagttttcaaggcaacttcctgcctgtggtttaatttgatagtcacaatgataggaaattggcagggttaggtggtggt
atctacattttgcccatgaggacttgtggctcagagaatttgagttttttctagtaaggtcactaggtccctagtgcacagcctgcttttaggt
acggatgtgggatgggggtgtcggcagaccacagggttttgactctcagtttgggctcatttccatgactccttgctgcctccctgatttct
gtcaaatgaccaaagcacatttagtaattacttctgctgcaaggactactccttattaggctgtgataagtaagtttcctcacatagtgggtc
agctcactctggccacaggacccagcttcctaaccacacacattaagaaagagaaaaattagcactgtctgagacctacaaccacttca
agggagaacagtggtgtttgcacaaatgtctacttttgttttaacctagtcatgtgtaaaaagtgtaattcactggtctttaccaaaaaaaaa
aaaaaaaaaacaacaacaaaaaaaaaacaggcatcatagtctgcctgtgctggacgctatgctagacactggagagacaaaggtga
gcaggataggcacaaccctacccttacaaacccagaaagttagaggaaaggccatcaggacattgctgtgcaatttgatatcatttcaa
ttttggtaacatagatatttgaactaattagaatgtgctacaaattttgccattctttactttccatatgccaacatggaaaatttgtaaccctcat
ctttaaatacctcatcttaccagtcctctataaatcaaaaaatctaccccaaaaagccctaggttttaacttttgaaaagtttttttttatcagctt
ttcaggtcatgactaagatctgccattcaatttaaaggaattctttcattttagagtaatagttactaaaatcctttattcctacctccttgagaat
ctaatggaaattttatatgctcttcccagaaaatttttgttaatgtgtatcctcacacaccaatttgcattcgatgttagcaggctagagacttc
cttgaatctcagccatgaccctcttcgggggtccactttcataatcagtattctaaacctagtaggcataaatagcttgaatagaggaaagt
ggtatgtagactcttgcccttcacttggactgggcacctctatcacttggactgggcaccagctagtgcccttaactagctggtgtctccat
ttttgatagtcattggctattgcttagctgaagctattgattttaacagccttattgttttgatatcaggctgccactctgcagaagtctaatact
ggggaaggagcaccggcctgagaaccatggtttgtttctaggaccaactctgcagatttctgtgcctcattttgctcatctctaagatggg
tggttgatgggtggcagggactgaaccatttctaaaatcccttacactgatgaaattctgtgattccagtgtaactaccaagctcactttcat
atacctcctgagacacagctggtcttagtgaatttatatcctctatgtccaaagtaagtcaaagtaagtaccactataattcactatagaatg
tatttttgtctttcactgatcttactggactcaaaactgttacatgtttccttaaattagatagaataaaaaaattgtgaaacactacttttcaagg
aaaaatattacaattttaaaagcctctttttatcccacctctcagtagctacaactttcatatacgagtttcacatatatatgaaaaaaattttctt
atctcagactcttaaggaaatgcaatgcttttaatatgatcactgctgtgtaattctcatctgccctgaaacacttgttctgtccatcaatcaat
gtatttgggtttacaatatgcagagcctcaagcaaaaccagtgcaaaagcacatctatcctcaaataggcacatcatatgtctgagcagtt
cagcatcctcactcacaagtccaaggctctcagcaatgtggaagaagatgtgattcctaaaattaccacccttacacacagtgacacact
caccagaaagtgaggcttaacatagcttaagtggctgtagtttaaaacagattgtgaatatctgtgctaattctgagaaacaattagcaga
catattaaccaattagcacagatcactaatatctgtgctaattgttaggacagattagcacagatattgctaccttgtgtgggtagacaggg
ttaaggcactggcagaaggaaagattagtgaggaatatcaaatagctgatttaatgaaattgtgaggccgatgtttggataaggtgaaac
agtgccctacaatgacatttatgccagttctccataaacaagagtgttctacacaaattataagttcctggctgtcttaacaaaatgctatgg
tcctttatatgctcacacaaataatggttccaagcatgggaaacaggtgccagattgcccaggtaggcatcccagcagcatgctccttag
ccactgattaaccttgcgcaagttatttcacctgcctgtgcctcaaggcccctgtgtttcaatggggagggtgtgaatactcattcctaagg
ttgtgaggaggaataactgagttagtatacgtcaaacacttaggtgcttggcacagagggagcctgtgtaagtgtttgctattattttgtttc
actagatgatcttgtgtattataagaaatcaatttggaaggtgagatacaaatgcagtctgagaaagtaaagacgttttaaagacattttact
gtgtcatgaattttcagagaccacactgacatggtaaaaagttgtttggtttttagaatttttgtttcagaatgcatatccttctccttaaccaaa
agaaaaactactaaaatacttctgaaaattatttccaagaaaacattttggtgtaccttttagcagttactttatgacttaaagattaggatgaa
caaaataattattttaacttggcttttacctgcttgtagataacctctaatttatataatggttcaattatacagtggtaaatgtatctgggcttgt
tcttttaaaatagcaagtgtaacatgtgatagatgtagggtacttacaaagtactcaccaagcattgatgactgatgtcattagtatcatggct
aacattcactgagccctgattatgaggctggcacccaactaaatgttttgcattgtcacttcatcttcacaaagactttttgaataggttattct
ttatctccattttcagagaagcaactaagccctaaagaggtttattaacttgccaaataccacacagctagtaacactggtggggtctgga
atggaacccaggcaagctgaaaacgacagaacaatgaacactcctacttttcaattcgatttgctaattgttaatatttgccacactggctt
atctctctgtatccctacccatctctctctctctctgtctatatctatatgcaaacctggcatctaacttctaaatgcttgagcacggatctccta
agaacaagtacatggttctatgcaatgaaaagacatgattatggcatccaagagtgattttaaaaatcaaggatgaaacttaatttttaccc
acaagtccagacgatgatttctaccagtattctccagctttactctgtttcttatacttgacagactgagctccaagcctggaatcttctaaaa
agggtccactgccaatctggaatgccaacaaatcttattaatatatctccacatttgtaagcttctactaaattctccaagaacctgcatatc
agttaagtcctgaaatgctccagtgccaaataaaagcccactgtgcagatagaaatgcttttggtgactctcaaacatttattcaaaggtat
ttagtgagtatctgtcagctgtatatccagcactgagggaagactcagtctctgccatcacatacacctcagcactaagaagagacacat
gaaataattaccatccagtgtgataagtgctctgacagaggatggagtgaggactagtcaccttggggaagacagagaagcagggtc
ccaccaggagatgagaagccagcctccaggagtgcgctatctgagagggcaagagaaggaaggcaaagaggacgggtccatact
tgaacaacttgaagtaggtgtatctagtgcctaagagtgtgtatggtgacatcttgtggagcagtgaacatgaccctcatttggtttgttaa
agcattgggtcaaatgccacgggaatgtaacaggtaaatggtctttgccttataacacagggtatcccatccgtgtaagacaatgaatttt
taaaaaggcaaaaccaaaccttttgtttacatcatttgccttaaatagttcttagtacacaaattcttccttgcttggggccattatggtgtgtg
gaagtagaaaaagataaactgtagcttaaaacaaacaggtaacacataataagttccaaatgaggcattttgttgaaattcagtgttgagc
ataaacagtgaactcaaatgcacagaggaggcgtcatggaagggtgacaaaacactccagagcaataacagctaccctattttctgttc
cctgggagtagggagaaaagacatcaaaatcaaattataaatttaaaaaagtattaaaattctcttatggaaatatattaagaagtttgttag
gcaaatggaaaacgttttgtggaaacaagaaataagaaagaatggccatcacttaaagcaacagaatgtatctctcccactagctctcc
cctccctacccacagctttattcacatttgttctataacatttcagcacagttcagaccttccgctgagccagtgtgaggtcagggtgttga
ggaagcgagtccctgcagtcagactccagctggctgtcgtcctgcttctgcttattccacagaatgagtctggtgggggagaggaaatt
cctaacctgtttttttttacacatcttcatcagaaaagcgaaagcactaattactgtccataataaaactactgggaaggttacttagcttgta
agtaagttccacaaagccatttcagtaaaaacttcctggttctaaaattgggaaaagcaggataatcctctaaggctgacattctttctctttt
gtgcatttacaactaaacggtcactgatacacatttccacagtgaccctgcaatatccacattggctttcttctaagacacttcaaaaataca
tcacacctttatgtgttaaatatcactattggcatatatctttaaaaacatttattttcttaattctcaaaatacagaaaagaatgttatgctattg
tgtgcacaaactgctattttcaataaggagctcttaatttagaaagttgctatttttcaataaagagctctcaattagaaaatatcaagaagtga
tgttaagtttttatggaaataaataaagtatcatgacatgaaaagtttataatttttcccccagcaatggcttactcagggaatttttcttatatgt
aaagttaatgatctgaaaagtggtaatcttatcattcaccaagatttcaggatataagttattcctacagaacaatccatttttacctttaaaga
tgagtttgtgtacttcagaagatgtgacgccagttggccaccatctcaatcagtagtaagtcacaccatcaatcccttgttaatgatggaac
tcatgtgaagtcaagaggaaacataatactctttacctcattccctggtagcagagaagttgactcatctttaatgtcagttattgagatgta
acacagaatcatattctacagaaaatatgtgataaactttacacaaatgaatacactttagaaaggcagtcatttggattttctttttgtcaga
gaacattgcaagtcttcctgaagaatttctgagaaattaaacactttatatgacattttacagcatgtagagaggttgaaactcagagaact
aaaactcagatgatagtctaaatacttaatttgttgctgctttatgctggacatgaaaagtgtgagcttaaattataaataaaaaactattgag
ctatcaaactatttggggtacatccttatctttcctctaaattactctttattcccattaaaatccacctatcccgggtcctctctcctcagccctt
cacctctgctttcaaagatgactaactaacctaataaacacatctaaggtgatgtgtcaacttcaaccccaaccctttttccccagATA
ACTTGTCTGCATTTCAAGAACAACCTACCAGAGACCTTACCTGTCACCTTGGCTC
TCCCACCCAATGGAGATGGCTCTAATGgtaaggaattctatcttaagtaaaagggaagggaaacacgctgac
agatgcagtttttataaaggcaggtcattggggcaccaagttggcagccgctttcaacttttgattctacctcaaaaggtaaccaagaaat
cgttgttattctaaagattatgacaagtccaaagtttcacagaatcctgatttttatacacttgcattaatcactgcttacttccatgatgtaaac
caaaactgcagcggcagaatatagagaatcagaataaattctgatagaaaaggcagatactgtttccacatatacaagtatatgctaatt
gcttttagcaaactgatccaaataggaatcaacattattccatcttaatgattatgtttttgattggtacacataaatgttaaagtgggaaaata
tgcagagaaagttacctaaactaaatcactttaattttcaacaacataattacattccatttcacctactaaaatggctttggacctagaatttg
tattgctacatcaatataagatgactttcagtaaaaatagatgtacagttgatttgtaacaacaaattaatagttttaaattatttgtatatttcaa
ggtaaataaaattagagctatcccacaatcgctttggttatgaaattcgaccactgtaactttttttaaagaaaactttatggtgtgcagttcct
ttggtatgcaaatattaaatggttggtgctgagaataagcagcaatatagaaatatatataattgtagaagaatataacctatgatttagaca
taaaatgagatccttacaagataaagagttatggagatggatgataatgatagttgtagaacattatgactaagtgtttaatatagcggacc
tgtacacttaaaaatggtgaagatgttaaacttacattatgtttacattacatatttcaccacaataaaaaatttttgaaggaaaaccagaact
ttaatgacaaacttaggacatattatgctctgaatatatagttttgataacgtctttcctggcaaatcaacaaggtaagaataaaaggatattc
tgaaatctttttttttctttggagacagagtcttgctctgttgcccaggctggagtgcagtggcacaatctcggctcactactaccttctgcct
cccaggttcaagtgatcctcctgcctcagcctcctgagtagctgggattataggcgtgcgccaccacgcctagctaatttttttgtattttta
gtagagacggggttttaccatgttggtcacgctggtctcaaactcctgacctcatgatccgcccacctcggcctcccaaaatgctaggat
tacaggcatgagacaccacacccagccagatattcttaaatcttatgctcacactccaccccccccaaaaaaattgtccttcaggaaagc
ataatttaaggaaaaatttcccaaagtgccaccttcagcttcattcttagcaccagaggaagaattgcctgtcatataatgaaagatccttg
gtgttatcattaatctagtatacatagtgcttcctcttggtagatttttacctactgtgagaaggaatgattttgatactcagacagtgtgtagta
atttgttaagaacatttaacttggaacctatgctacacagtaaaacttatactgacaatggatatttcatgtggtaggcctaagaagtattgtg
gagtgaggggactggaagtatatctgcagaactacctagtcaaagaccccagtctaatttctaatatataattcatataaattactcttgcat
tgctatacattaaacattatgcttgtcacattcttacttggttagcactgtcactactgattgtcaggcctataattgtgtataattcttcccctgc
cactttaccagctggtggccacaccgattccagaaagaactactttatttcttattacctcatgaaagcaccctctgcccagtgccttggct
aatagactggcctttgttgtctatgaccactaattcccactcaggcatatagcaattctgttgttggttgcttagcccagtcaaaaacactgg
aagtggacatagactccacactgatgaatgaatgaccttctagtcattctaatgtccttaaaagtctactatttttaaattgtagaatatatata
tatatatatatatttttttttttttctgggatatttctcctttcttctgttcattaatgtttttcatctatcattgtgttttaaatatgacctaagg
ttctatagtatatttagacaaacttctcatttctctgcagtgttcggaattcatcatgcctgtatgacaaggttgtgtttgagaacaaaaggggacc
tgtgtgacatgttttatttcaatatacatttagagtttgaacaaataaaaaaagctattagaatttttaatatatataacacattatcaaaaacact
gtcacttttctgagtgctctaatcaggcaattcgtatgtatatttaaaaagagggaaaaagctaattagtatactaaatatatttattttaatacct
gtgctcatatgttatctttaaagaacaggaataaaatttatgggtagaatagtatgccagcaatttaccttcaatacaaattaaacacatcttttaa
gaaaagggcgagttatattggctggggtggcactaacactacattattaatatgtatttattgagcatttactgtgtccaggaccctgtaag
ggggaaaaaaagtccttgtttttacaatctttattgactgaaattctgtaaaatgaattgaattcctgaagtgacatcatttaacccaaatttttt
catcatcaactaattcatctcagaaaacagtaaagtttaaaaaaaaatactatcagtaagaataatatatttccttggtttggaaatttccaaa
ccacgatattctgccctgtcaaaatatattcactggggaattttttacttcatacgtttcttttgaacttagtcacatttttatatctagctggttt
cattattaaataaaagaaaaggtgattcccctcttgtactgcaggagaccacatacttgaatgatactctaacttctaggttctgttatagtaac
actgaaatcagaaacctgtgggagaaatgtcactttatctgagagagtaaaatcagactctaaaaggaagtagcaaattcatcttgttcttt
tttttaatcaaccctctgcctaagacaagtagtttgaaacacagaggactctttaagtcatacttcctttatgtagtcaaaaccaaagccact
gggtagtgctttccccaaaggaatctctaaatagtagacggggacattttcagatagattcgtttgtaggcaaacctccattgcttgtatca
catttcctgaaagaataaaggtaaaacttcaactatgtattacagaaagaaaaattcagcctgaaccctacccttataaaacaggttaattg
ggttttaattttcataaatcataaaggactattttgaacatttgggcctttaattgtctagctcctagatgaagtacaaatcagaaaaaaaaaa
aactgtactgtgtcagaatgcaagctttcctctttgcattttggcatttgaaaactccgaagagcggtttttgttttttatttaaagaagatgata
catatgtgtacccgattcaaaactagagaatagaatttaaaacataattttcaaagtcttcaaatatgcctaaaggtaacaatgtcatctttta
attgccaatttctctaccactttcaaaaaattacttccaaggatttaatgagctccttcctttcaacagaaaatggactattttcctttcagattta
ctatatgctgtcactccagctttataaccgcatgtgcatacacaaacatttctttctctcttgcagGTGGCACAAACCAGGA
AGGGGAAATCTGTGGTTTAAATTCTTTATGCCTCATCCTCTGAGTGCTGAAGGCT
TGCTGTAGGCTGTATGCTGTTAATGCTAATCGTGATAGGGGTTTTTGCCTCCAACT
GACTCCTACATATTAGCATTAACAGTGTATGATGCCTGTTACTAGCATTCACATG
GAACAAATTGCTGCCGTGGGAGGATGACAAAGAAGCATGAGTCACCCTGCTGGA
TAAACTTAGACTTCAGGCTTTATCATTTTTCAATCTGTTAATCATAATCTGGTCAC
TGGGATGTTCAACCTTAAACTAAGTTTTGAAAGTAAGGTTATTTAAAAGATTTAT
CAGTAGTATCCTAAATGCAAACATTTTCATTTAAATGTCAAGCCCATGTTTGTTTT
TATCATTAACAGAAAATATATTCATGTCATTCTTAATTGCAGGTTTTGGCTTGTTC
ATTATAATGTTCATAAACACCTTTGATTCAACTGTTAGAAATGTGGGCTAAACAC
AAATTTCTATAATATTTTTGTAGTTAAAAATTAGAAGGACTACTAACCTCCAGTT
ATATCATGGATTGTCTGGCAACGTTTTTTAAAAGATTTAGAAACTGGTACTTTCCC
CCAGGTAACGATTTTCTGTTCAGGCAACTTCAGTTTAAAATTAATACTTTTATTTG
ACTCTTAAAGGGAAACTGAAAGGCTATGAAGCTGAATTTTTTTAATGAAATATTT
TTAACAGTTAGCAGGGTAAATAACATCTGACAGCTAATGAGATATTTTTTCCATA
CAAGATAAAAAGATTTAATCAAAAAATTTCATATTTGAAATGAAGTCCCAAATCT
AGGTTCAAGTTCAATAGCTTAGCCACATAATACGGTTGTGCGAGCAGAGAATCT
ACCTTTCCACTTCTAAGCCTGTTTCTTCCTCCATATGGGGATAATACTTTACAAGG
TTGTTGTGAGGCTTAGATGAGATAGAGAATTATTCCATAAGATAATCAAGTGCTA
CATTAATGTTATAGTTAGATTAATCCAAGAACTAGTCACCCTACTTTATTAGAGA
AGAGAAAAGCTAATGATTTGATTTGCAGAATATTTAAGGTTTGGATTTCTATGCA
GTTTTTCTAAATAACCATCACTTACAAATATGTAACCAAACGTAATTGTTAGTAT
ATTTAATGTAAACTTGTTTTAACAACTCTTCTCAACATTTTGTCCAGGTTATTCAC
TGTAACCAAATAAATCTCATGAGTCTTTAGTTGATTTAAAATAA

We claim:

1. A genetically modified T cell comprising a nucleic acid sequence encoding a chimeric antigen receptor (CAR) polypeptide comprising a single-chain variable fragment (scFV) that specifically binds to a target molecule, wherein the nucleic acid sequence encoding the CAR is integrated into an integration site located at a miR-155 host gene.

2. The genetically modified T cell of claim 1, wherein the integration site is at exon 1, exon 2, exon 3, or a transcriptional start site of the miR-155 host gene.

3. The genetically modified T cell of claim 2, wherein the integration site is at exon 3 of the miR-155 host gene.

4. The genetically modified T cell of claim 1, further comprising:
a ribonucleoprotein (RNP) complex comprising a CRISPR/Cas endonuclease (Cas9) system complexed with one or more guide RNAs targeting the miR-155 host gene or a fragment thereof; and
an AAV vector comprising a polynucleotide sequence encoding the CAR polypeptide; wherein the polynucleotide sequence is flanked by a first homology arm and a second homology arm.

5. The genetically modified T cell of claim 4, wherein the first homology arm and the second homology arm comprise a sequence at least 80% identical to SEQ ID NO: 3 or 4, or a fragment thereof.

6. The genetically modified T cell of claim 4, wherein the CRISPR/Cas9 system comprises a first guide RNA and a second guide RNA.

7. The genetically modified T cell of claim 6, wherein the first guide RNA comprises a polynucleotide sequence at least 80% identical to SEQ ID NO: 1 or a fragment thereof.

8. The genetically modified T cell of claim 6, wherein the second guide RNA comprises a polynucleotide sequence at least 80% identical to SEQ ID NO: 2 or a fragment thereof.

9. The genetically modified T cell of claim 1, wherein the target molecule is a cancer-related protein.

10. The genetically modified T cell of claim 9, wherein the cancer-related protein is CD19, GD2, or CD33.

11. The genetically modified T cell of claim 4, wherein the AAV vector comprises a polynucleotide sequence at least 80% identical to SEQ ID NO: 6 or a fragment thereof.

12. The genetically modified T cell of claim 1, wherein the T cell is a primary T cell, a T cell line, a tumor infiltrating lymphocyte, an effector T cell, a memory T cell, a TEMRA, or a stem cell-like memory T cell.

13. A method for treating cancer in a subject, comprising administering to the subject a therapeutically effective amount of the genetically modified T cell of claim 1.

14. The method of claim 13, wherein the T cell is cultured ex vivo for at least 2 days prior to administration to the subject.

15. The method of claim 13, wherein the T cell is cultured ex vivo for at least 7 days prior to administration to the subject.

16. The method of claim 13, wherein the T cell is cultured with IL-2, IL-7, or IL-15, or any combination thereof.

17. The method of claim 13, wherein the T cell is derived from the subject.

18. The method of claim 13, wherein administration of the genetically modified T cell prevents or treats acute graft-versus-host disease.

19. A gene-editing system for engineering a CAR-T cell, said system comprising:
a ribonucleoprotein (RNP) complex comprising a CRISPR/Cas endonuclease (Cas9) system complexed with one or more guide RNAs targeting a miR-155 host gene or a fragment thereof; and
an AAV vector comprising a polynucleotide sequence encoding a CAR polypeptide; wherein the polynucleotide sequence is flanked by a first homology arm and a second homology arm.

20. A method of creating a genetically modified T cell comprising a nucleic acid sequence encoding a chimeric antigen receptor (CAR) polypeptide integrated into an integration site located at a miR-155 host gene, said method comprising:
obtaining a T cell; and
introducing into the T cell the gene-editing system of claim 19, thereby creating the genetically modified T cell.


SEQUENCE LISTING (8 sequences)

SEQ ID NO: 1 - RNA, 20 nt, Synthetic construct
  source 1..20: mol_type = other RNA
tcttgcaggtggcacaaacc

SEQ ID NO: 2 - RNA, 20 nt, Synthetic construct
  source 1..20: mol_type = other RNA
atggaacaaattgctgccgt

SEQ ID NO: 3 - DNA, 600 bp, synthetic construct
  source 1..600: mol_type = other DNA
ggggacattttcagatagattcgtttgtaggcaaacctccattgcttgtatcacatttcctgaaagaataaaggtaaaacttcaactatgtattacagaaagaaaaattcagcctgaaccctacccttataaaacaggttaattgggttttaattttcataaatcataaaggactattttgaacatttgggcctttaattgtctagctcctagatgaagtacaaatcagaaaaaaaaaaaactgtactgtgtcagaatgcaagctttcctctttgcattttggcatttgaaaactccgaagagcggtttttgttttttatttaaagaagatgatacatatgtgtacccgattcaaaactagagaatagaatttaaaacataattttcaaagtcttcaaatatgcctaaaggtaacaatgtcatcttttaattgccaatttctctaccactttcaaaaaattacttccaaggatttaatgagctccttcctttcaacagaaaatggactattttcctttcagatttactatatgctgtcactccagctttataaccgcatgtgcatacacaaacatttctttctctcttgcaggtggcaca

SEQ ID NO: 4 - DNA, 600 bp, synthetic construct
  source 1..600: mol_type = other DNA
ccgtgggaggatgacaaagaagcatgagtcaccctgctggataaacttagacttcaggctttatcatttttcaatctgttaatcataatctggtcactgggatgttcaaccttaaactaagttttgaaagtaaggttatttaaaagatttatcagtagtatcctaaatgcaaacattttcatttaaatgtcaagcccatgtttgtttttatcattaacagaaaatatattcatgtcattcttaattgcaggttttggcttgttcattataatgttcataaacacctttgattcaactgttagaaatgtgggctaaacacaaatttctataatatttttgtagttaaaaattagaaggactactaacctccagttatatcatggattgtctggcaacgttttttaaaagatttagaaactggtactttcccccaggtaacgattttctgttcaggcaacttcagtttaaaattaatacttttatttgactcttaaagggaaactgaaaggctatgaagctgaatttttttaatgaaatatttttaacagttagcagggtaaataacatctgacagctaatgagatattttttccatacaag

SEQ ID NO: 5 - DNA, 147 bp, synthetic construct
  source 1..147: mol_type = other DNA
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattc

SEQ ID NO: 6 - DNA, 6346 bp, synthetic construct
  source 1..6346: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggcgcgccggggacattttcagatagattcgtttgtaggcaaacctccattgcttgtatcacatttcctgaaagaataaaggtaaaacttcaactatgtattacagaaagaaaaattcagcctgaaccctacccttataaaacaggttaattgggttttaattttcataaatcataaaggactattttgaacatttgggcctttaattgtctagctcctagatgaagtacaaatcagaaaaaaaaaaaactgtactgtgtcagaatgcaagctttcctctttgcattttggcatttgaaaactccgaagagcggtttttgttttttatttaaagaagatgatacatatgtgtacccgattcaaaactagagaatagaatttaaaacataattttcaaagtcttcaaatatgcctaaaggtaacaatgtcatcttttaattgccaatttctctaccactttcaaaaaattacttccaaggatttaatgagctccttcctttcaacagaaaatggactattttcctttcagatttactatatgctgtcactccagctttataaccgcatgtgcatacacaaacatttctttctctcttgcaggtggcacacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcatcgatcacgagactagcctcgagaagcttgatatcgaattccacggggttggacgcgtcttaattaaggatccaaggtcaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcaccgccaccatgctgctgctggtgacctctctgctgctgtgcgagctgccacacccagccttcctgctgatcccagacatccagatgacacagagccccagctccctgagcgcctccgtgggcgacagagtgaccatcacatgtagggcctctgagagcgtggataactatggcatcagcttcatgaattggtttcagcagaagcctggcggcgccccaaagctgctgatctacgcagccagcatgcagggctccggcgtgccctctcggttctccggctctggcagcggcaccgacttcaccctgacaatctctagcctgcagccagacgatttcgccacatactattgccagcagagcaaggaggtgccctggacctttggccagggcacaaaggtggagatcaagggctccacctctggcagcggcaagcctggcagcggagagggctccacaaagggacaggtgcagctggtgcagtccggagccgaggtgaagaagccaggctcctctgtgaaggtgtcttgtaaggccagcggctataccttcacagactacaacatgcactgggtgcgccaggcaccaggacagggcctggagtggatcggctacatctatccttacaacggcggcaccggctataatcagaagtttaagtccaaggccaccatcacagccgatgagtctaccaatacagcctacatggagctgagcagcctgcggtccgaggacacagccgtgtactattgcgcccggggcagacccgctatggactattggggccagggcaccctggtgacagtgtctagcacaacaactccagcaccccgcccacctactcccgctcctacaatcgcctctcagcccctgtccctgcgcccagaagcctgtcgccccgcagcaggaggagcagtgcacaccaggggcctggacttcgcctgcgatatctacatctgggctcctctggctggaacttgtggcgtgctgctgctgtcactggtcattaccctgtattgtaagcggggcagaaagaagctgctgtacatcttcaagcagccctttatgaggcctgtgcagaccacacaggaggaggacggctgcagctgtcggttccccgaggaggaggagggcggctgcgagctgcgggtgaagtttagcagatccgccgatgcaccagcatatcagcagggacagaatcagctgtacaacgagctgaatctgggcaggcgcgaggagtacgacgtgctggataagaggcggggccgggaccccgagatgggaggcaagcccaggcgcaagaaccctcaggagggcctgtataatgagctgcagaaggacaagatggccgaggcctacagcgagatcggcatgaagggagagcggagaaggggcaagggacacgatggcctgtatcagggcctgtccaccgccacaaaggacacctacgatgcactgcacatgcaggccctgccacctcggtgaaagtaacgccctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcccgtgggaggatgacaaagaagcatgagtcaccctgctggataaacttagacttcaggctttatcatttttcaatctgttaatcataatctggtcactgggatgttcaaccttaaactaagttttgaaagtaaggttatttaaaagatttatcagtagtatcctaaatgcaaacattttcatttaaatgtcaagcccatgtttgtttttatcattaacagaaaatatattcatgtcattcttaattgcaggttttggcttgttcattataatgttcataaacacctttgattcaactgttagaaatgtgggctaaacacaaatttctataatatttttgtagttaaaaattagaaggactactaacctccagttatatcatggattgtctggcaacgttttttaaaagatttagaaactggtactttcccccaggtaacgattttctgttcaggcaacttcagtttaaaattaatacttttatttgactcttaaagggaaactgaaaggctatgaagctgaatttttttaatgaaatatttttaacagttagcagggtaaataacatctgacagctaatgagatattttttccatacaaggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 7 - DNA, 147 bp, synthetic construct; identical to SEQ ID NO: 5
  source 1..147: mol_type = other DNA

SEQ ID NO: 8 - DNA, 16210 bp, synthetic construct
  source 1..16210: mol_type = other DNA
gatcaagatgtcagcaggactggttcctcctgagagctgtgagggaaggatctgtttcaagcccctctccttggctggtagatggccatcttttcccaatgtttcttcacactgtcttccttctatatgtgtttctttctctacacatcatgttctttttctaagaacaccagtcatattggattaggagccaaccctattttaataagacctcatcttgactaattacatccacaaaggccttgtttccacataaggttatattctgaagtactggttggtctacagccataccaccccacacacacccgatctcatctgaagtactgggtgttaggacatcaatatatgaatttgggaggtggggggagggggacacaattcagcccacaacaacatccatggggtacttgtcccggcaggtggccagctcttactttctcaagctatccatttggagcaggtccttttttttttttctttttttttttttttgagacagtctcgctcgcattccaggctggaatgcagtggcgcgatcttggctcaccgcaaactccgcctcccgcattcatgccattctcctgcctcagcctccagagtagctgggactacaggtgcccgccatcatgcccggctaatttttttttcttttttgtatttttagtagattcagggtttcaccattctagccaggatggtctcaatctcctgacctcatgatccagtcacctcagcctcccaaagtgctgggattacaggatgagccaccgcgcctggcctggagcaggtactttttatccatcaccttatttaatcatcatatccctcttagtctgctagggttgccataagaaaataccatagactggatggctgatacaacaacaatttattttctcacattctggaggctagaaagttccagatcaaggtcctgcagggttcggtttctggtgaggactctcttcctggcttacaggaaaccaccatcccattgtgtgctcacatgacctctgctttgactgatttctgagagaaagggaattctggtgtctcttcctcttctcataagagcactagctctatgggattagtgatccacccttttgacctcatttaacctttatcacctcctcacaggccctatctccaaatggggataacattggggatcagggcttcaacatataaagttgaagagaacatgattcagtctatagtaatccctgatgggagacatcattattgtcatttacaggaagctgagacataaaaagtttaacgttcagattccacagctagaattagtgtaaaatgcaggtgtatttgactcctaaaacccatttatccatccactattctgggtgtctcaaactgggacctaaaatgcggaggtttgcacatgacctgaaattacttccaccactgggagaagggaggattgagcaatttcctgtttctattaaagtgcattatacaaacttgtcattctggggatgaaaggtcaccctagaattgcctatgggcaatttcttatagttcaacctagaatgagaaatgggaaattcagaaaggcattgtaggcatctgtaaccagcagagggacgtgccccacctgggtggggaccatgcatccttgccacatgccccactgcacaacttccccagctcctcaacgtcacatggatctggaaagcagggagactggactacggagccagccctccagggttggaactgagtttgaatctcagcttgactacttactaggatctaaggtaacatgtttaatctctctgtgctcagtttcctcatctggaaatagagattataatgctcctccctcatatgatcatagtgaggactgaaagagttaatgcatataaggagtgttaggacaggatctggcacatggtaaatgctttataagtgttagttgttatcatcatgattattcttgtttgaattataagagaaaaagcatgtatcttaagaatagagggttttaaaatgctcccaagttccttaaccaacctgagccatctgtaaattaagtactatgggatttccagctctgacatgtattctgacatgtaactggcatcagtttaatacaaactattctaaaatgtcttgtgcccttgacagggaggtccaagtactggatacttgcaatgcaatccagcagtggccccgtctttcttacaaaaaggccccagtcacatgttgatgaggctagatctattcctgtcctccttctcttccccatatttccttattcttcttcaagtctaaacgtttattgaactgagacccatgaatgagttactcgactaggcttgtaggataaacttgccaggctttagatctatttgttcctttccatcccccaaaatcaaatggctgctccaggaaaatgttccccttgtggcagggtccgggagaaaagagagaagcgacaaaaccaaaaattaaaacgaccgaagtcccatatgctccaggaatatgtcctggagatgggagtggagggcagggggagaatgttgttgaggtcaaaatttttgaagttttaagtcctatatcttgacatcccgagtataaatgcgggtaccagacacagtacaaacgttctcaaagcccagttacgtattccaaaccaaacgcgggctcttgaagggtgatgaggtagggatgaaatccaggatcgcctgaagaccatttcttcctctcttagggacctgctggtctccagctgattcggtccaggaggaaaaacctcccacttgctcctctcgggctccctgcaaggagagagtagagacactcctgccacccagttgcaagaagtcgccacttccccctccagccgactgaaagttcgggcgacgtctgggccgtcatttgaaggcgtttccttttctttaagaacaaaggttggagcccaagccttgcggcgcggtgcaggaaagtacacggcgtgtgttgagagaaaaaaaatacacacacgcaatgacccacgagaaagggaaaggggaaaacaccaactacccgggcgctgggctttttcgacttttcctttaaaaagaaaaaagtttttcaagctgtaggttccaagaacaggcaggaggggggagaagggggggggggttgcagaaaaggcgcctggtcggttatgagtcacaagtgagttataaaagggtcgcacgttcgcaggcgcgggcttcctgtgcgcggccgagcccgggcccagcgccgcctgcagcctcgggaagggagcggatagcggagccccgagccgcccgcagagcaagcgcggggaaccaaggagacgctcctggcactgcaggtacgccgacttcagtctcgcgctcccgcccgcctttcctctcttgaacgtggcagggacgccgggggacttcggtgcgagggtcaccgccgggttaactggcgaggcaaggcgggggcagcgcgcacgtggccgtggagcccggcctggtcccgcgcgcgcctgcgggtgccccctggggactcagtggtgtcgcctcgcccgggaccagagattgcgctggatggattcccgcgggcagaggcagggggaaggaggggtgttcgaaacctaatacttgagcttctttgcaaagtttccttggatggttggggacgtacctgtataatggccctggaccagcttccctgttggagtggccagagaagtgtgtaaaacacactagaggggcagggtggaaaaagagactgccttcaaaacttgtatcttttcgatttcattttgaaaaataactacaaatctattttaattttacaaagttagactcatagcattttagatatcaatgtcttcatttaacagaagtgaagatggagcaaacgctcaatcagcgtctgtatttattcgctcctgttgtgccagggtgcgtttttgccgagcggttgcctttctttactcacaaaacccccttgatgtctgtcctccacgttttacgagggagagccggatcttttgaagtttgtatcatctaaagcaggtatattgggatgactatggatagaatttaacctgaaaacactgaagttgacagctgacaaagatataagaatccaaagtatgttaaaaattaaggagctgaggcccacagaagtgaagttacttttccagcatcacacagcagatctaggaccctgtgtgatttgaaaaggccaaggtatctaatgatgctcataatttagtttacattttgtgtcaaatagatgaggtcaagtggacattatgtgtatattccaaagccaaaattactgcttgtaatatccattcatctctccgaagacgtagtgcctcttttatatggctttaacctaatgtataatcgaagtgaattagggcagccagacttttaatcacaggactgatcttttaaaaacgggtatatgtaaagttaattgcttatctatgtgaacagtaatatacactataaaaagttttcaaggcaacttcctgcctgtggtttaatttgatagtcacaatgataggaaattggcagggttaggtggtggtatctacattttgcccatgaggacttgtggctcagagaatttgagttttttctagtaaggtcactaggtccctagtgcacagcctgcttttaggtacggatgtgggatgggggtgtcggcagaccacagggttttgactctcagtttgggctcatttccatgactccttgctgcctccctgatttctgtcaaatgaccaaagcacatttagtaattacttctgctgcaaggactactccttattaggctgtgataagtaagtttcctcacatagtgggtcagctcactctggccacaggacccagcttcctaaccacacacattaagaaagagaaaaattagcactgtctgagacctacaaccacttcaagggagaacagtggtgtttgcacaaatgtctacttttgttttaacctagtcatgtgtaaaaagtgtaattcactggtctttaccaaaaaaaaaaaaaaaaaaacaacaacaaaaaaaaaacaggcatcatagtctgcctgtgctggacgctatgctagacactggagagacaaaggtgagcaggataggcacaaccctacccttacaaacccagaaagttagaggaaaggccatcaggacattgctgtgcaatttgatatcatttcaattttggtaacatagatatttgaactaattagaatgtgctacaaattttgccattctttactttccatatgccaacatggaaaatttgtaaccctcatctttaaatacctcatcttaccagtcctctataaatcaaaaaatctaccccaaaaagccctaggttttaacttttgaaaagtttttttttatcagcttttcaggtcatgactaagatctgccattcaatttaaaggaattctttcattttagagtaatagttactaaaatcctttattcctacctccttgagaatctaatggaaattttatatgctcttcccagaaaatttttgttaatgtgtatcctcacacaccaatttgcattcgatgttagcaggctagagacttccttgaatctcagccatgaccctcttcgggggtccactttcataatcagtattctaaacctagtaggcataaatagcttgaatagaggaaagtggtatgtagactcttgcccttcacttggactgggcacctctatcacttggactgggcaccagctagtgcccttaactagctggtgtctccatttttgatagtcattggctattgcttagctgaagctattgattttaacagccttattgttttgatatcaggctgccactctgcagaagtctaatactggggaaggagcaccggcctgagaaccatggtttgtttctaggaccaactctgcagatttctgtgcctcattttgctcatctctaagatgggtggttgatgggtggcagggactgaaccatttctaaaatcccttacactgatgaaattctgtgattccagtgtaactaccaagctcactttcatatacctcctgagacacagctggtcttagtgaatttatatcctctatgtccaaagtaagtcaaagtaagtaccactataattcactatagaatgtatttttgtctttcactgatcttactggactcaaaactgttacatgtttccttaaattagatagaataaaaaaattgtgaaacactacttttcaaggaaaaatattacaattttaaaagcctctttttatcccacctctcagtagctacaactttcatatacgagtttcacatatatatgaaaaaaattttcttatctcagactcttaaggaaatgcaatgcttttaatatgatcactgctgtgtaattctcatctgccctgaaacacttgttctgtccatcaatcaatgtatttgggtttacaatatgcagagcctcaagcaaaaccagtgcaaaagcacatctatcctcaaataggcacatcatatgtctgagcagttcagcatcctcactcacaagtccaaggctctcagcaatgtggaagaagatgtgattcctaaaattaccacccttacacacagtgacacactcaccagaaagtgaggcttaacatagcttaagtggctgtagtttaaaacagattgtgaatatctgtgctaattctgagaaacaattagcagacatattaaccaattagcacagatcactaatatctgtgctaattgttaggacagattagcacagatattgctaccttgtgtgggtagacagggttaaggcactggcagaaggaaagattagtgaggaatatcaaatagctgatttaatgaaattgtgaggccgatgtttggataaggtgaaacagtgccctacaatgacatttatgccagttctccataaacaagagtgttctacacaaattataagttcctggctgtcttaacaaaatgctatggtcctttatatgctcacacaaataatggttccaagcatgggaaacaggtgccagattgcccaggtaggcatcccagcagcatgctccttagccactgattaaccttgcgcaagttatttcacctgcctgtgcctcaaggcccctgtgtttcaatggggagggtgtgaatactcattcctaaggttgtgaggaggaataactgagttagtatacgtcaaacacttaggtgcttggcacagagggagcctgtgtaagtgtttgctattattttgtttcactagatgatcttgtgtattataagaaatcaatttggaaggtgagatacaaatgcagtctgagaaagtaaagacgttttaaagacattttactgtgtcatgaattttcagagaccacactgacatggtaaaaagttgtttggtttttagaatttttgtttcagaatgcatatccttctccttaaccaaaagaaaaactactaaaatacttctgaaaattatttccaagaaaacattttggtgtaccttttagcagttactttatgacttaaagattaggatgaacaaaataattattttaacttggcttttacctgcttgtagataacctctaatttatataatggttcaattatacagtggtaaatgtatctgggcttgttcttttaaaatagcaagtgtaacatgtgatagatgtagggtacttacaaagtactcaccaagcattgatgactgatgtcattagtatcatggctaacattcactgagccctgattatgaggctggcacccaactaaatgttttgcattgtcacttcatcttcacaaagactttttgaataggttattctttatctccattttcagagaagcaactaagccctaaagaggtttattaacttgccaaataccacacagctagtaacactggtggggtctggaatggaacccaggcaagctgaaaacgacagaacaatgaacactcctacttttcaattcgatttgctaattgttaatatttgccacactggcttatctctctgtatccctacccatctctctctctctctgtctatatctatatgcaaacctggcatctaacttctaaatgcttgagcacggatctcctaagaacaagtacatggttctatgcaatgaaaagacatgattatggcatccaagagtgattttaaaaatcaaggatgaaacttaatttttacccacaagtccagacgatgatttctaccagtattctccagctttactctgtttcttatacttgacagactgagctccaagcctggaatcttctaaaaagggtccactgccaatctggaatgccaacaaatcttattaatatatctccacatttgtaagcttctactaaattctccaagaacctgcatatcagttaagtcctgaaatgctccagtgccaaataaaagcccactgtgcagatagaaatgcttttggtgactctcaaacatttattcaaaggtatttagtgagtatctgtcagctgtatatccagcactgagggaagactcagtctctgccatcacatacacctcagcactaagaagagacacatgaaataattaccatccagtgtgataagtgctctgacagaggatggagtgaggactagtcaccttggggaagacagagaagcagggtcccaccaggagatgagaagccagcctccaggagtgcgctatctgagagggcaagagaaggaaggcaaagaggacgggtccatacttgaacaacttgaagtaggtgtatctagtgcctaagagtgtgtatggtgacatcttgtggagcagtgaacatgaccctcatttggtttgttaaagcattgggtcaaatgccacgggaatgtaacaggtaaatggtctttgccttataacacagggtatcccatccgtgtaagacaatgaatttttaaaaaggcaaaaccaaaccttttgtttacatcatttgccttaaatagttcttagtacacaaattcttccttgcttggggccattatggtgtgtggaagtagaaaaagataaactgtagcttaaaacaaacaggtaacacataataagttccaaatgaggcattttgttgaaattcagtgttgagcataaacagtgaactcaaatgcacagaggaggcgtcatggaagggtgacaaaacactccagagcaataacagctaccctattttctgttccctgggagtagggagaaaagacatcaaaatcaaattataaatttaaaaaagtattaaaattctcttatggaaatatattaagaagtttgttaggcaaatggaaaacgttttgtggaaacaagaaataagaaagaatggccatcacttaaagcaacagaatgtatctctcccactagctctcccctccctacccacagctttattcacatttgttctataacatttcagcacagttcagaccttccgctgagccagtgtgaggtcagggtgttgaggaagcgagtccctgcagtcagactccagctggctgtcgtcctgcttctgcttattccacagaatgagtctggtgggggagaggaaattcctaacctgtttttttttacacatcttcatcagaaaagcgaaagcactaattactgtccataataaaactactgggaaggttacttagcttgtaagtaagttccacaaagccatttcagtaaaaacttcctggttctaaaattgggaaaagcaggataatcctctaaggctgacattctttctcttttgtgcatttacaactaaacggtcactgatacacatttccacagtgaccctgcaatatccacattggctttcttctaagacacttcaaaaatacatcacacctttatgtgttaaatatcactattggcatatatctttaaaaacatttattttcttaattctcaaaatacagaaaagaatgttatgctattgtgtgcacaaactgctattttcaataaggagctcttaatttagaaagttgctatttttcaataaagagctctcaattagaaaatatcaagaagtgatgttaagtttttatggaaataaataaagtatcatgacatgaaaagtttataatttttcccccagcaatggcttactcagggaatttttcttatatgtaaagttaatgatctgaaaagtggtaatcttatcattcaccaagatttcaggatataagttattcctacagaacaatccatttttacctttaaagatgagtttgtgtacttcagaagatgtgacgccagttggccaccatctcaatcagtagtaagtcacaccatcaatcccttgttaatgatggaactcatgtgaagtcaagaggaaacataatactctttacctcattccctggtagcagagaagttgactcatctttaatgtcagttattgagatgtaacacagaatcatattctacagaaaatatgtgataaactttacacaaatgaatacactttagaaaggcagtcatttggattttctttttgtcagagaacattgcaagtcttcctgaagaatttctgagaaattaaacactttatatgacattttacagcatgtagagaggttgaaactcagagaactaaaactcagatgatagtctaaatacttaatttgttgctgctttatgctggacatgaaaagtgtgagcttaaattataaataaaaaactattgagctatcaaactatttggggtacatccttatctttcctctaaattactctttattcccattaaaatccacctatcccgggtcctctctcctcagcccttcacctctgctttcaaagatgactaactaacctaataaacacatctaaggtgatgtgtcaacttcaaccccaaccctttttccccagataacttgtctgcatttcaagaacaacctaccagagaccttacctgtcaccttggctctcccacccaatggagatggctctaatggtaaggaattctatcttaagtaaaagggaagggaaacacgctgacagatgcagtttttataaaggcaggtcattggggcaccaagttggcagccgctttcaacttttgattctacctcaaaaggtaaccaagaaatcgttgttattctaaagattatgacaagtccaaagtttcacagaatcctgatttttatacacttgcattaatcactgcttacttccatgatgtaaaccaaaactgcagcggcagaatatagagaatcagaataaattctgatagaaaaggcagatactgtttccacatatacaagtatatgctaattgcttttagcaaactgatccaaataggaatcaacattattccatcttaatgattatgtttttgattggtacacataaatgttaaagtgggaaaatatgcagagaaagttacctaaactaaatcactttaattttcaacaacataattacattccatttcacctactaaaatggctttggacctagaatttgtattgctacatcaatataagatgactttcagtaaaaatagatgtacagttgatttgtaacaacaaattaatagttttaaattatttgtatatttcaaggtaaataaaattagagctatcccacaatcgctttggttatgaaattcgaccactgtaactttttttaaagaaaactttatggtgtgcagttcctttggtatgcaaatattaaatggttggtgctgagaataagcagcaatatagaaatatatataattgtagaagaatataacctatgatttagacataaaatgagatccttacaagataaagagttatggagatggatgataatgatagttgtagaacattatgactaagtgtttaatatagcggacctgtacacttaaaaatggtgaagatgttaaacttacattatgtttacattacatatttcaccacaataaaaaatttttgaaggaaaaccagaactttaatgacaaacttaggacatattatgctctgaatatatagttttgataacgtctttcctggcaaatcaacaaggtaagaataaaaggatattctgaaatctttttttttctttggagacagagtcttgctctgttgcccaggctggagtgcagtggcacaatctcggctcactactaccttctgcctcccaggttcaagtgatcctcctgcctcagcctcctgagtagctgggattataggcgtgcgccaccacgcctagctaatttttttgtatttttagtagagacggggttttaccatgttggtcacgctggtctcaaactcctgacctcatgatccgcccacctcggcctcccaaaatgctaggattacaggcatgagacaccacacccagccagatattcttaaatcttatgctcacactccaccccccccaaaaaaattgtccttcaggaaagcataatttaaggaaaaatttcccaaagtgccaccttcagcttcattcttagcaccagaggaagaattgcctgtcatataatgaaagatccttggtgttatcattaatctagtatacatagtgcttcctcttggtagatttttacctactgtgagaaggaatgattttgatactcagacagtgtgtagtaatttgttaagaacatttaacttggaacctatgctacacagtaaaacttatactgacaatggatatttcatgtggtaggcctaagaagtattgtggagtgaggggactggaagtatatctgcagaactacctagtcaaagaccccagtctaatttctaatatataattcatataaattactcttgcattgctatacattaaacattatgcttgtcacattcttacttggttagcactgtcactactgattgtcaggcctataattgtgtataattcttcccctgccactttaccagctggtggccacaccgattccagaaagaactactttatttcttattacctcatgaaagcaccctctgcccagtgccttggctaatagactggcctttgttgtctatgaccactaattcccactcaggcatatagcaattctgttgttggttgcttagcccagtcaaaaacactggaagtggacatagactccacactgatgaatgaatgaccttctagtcattctaatgtccttaaaagtctactatttttaaattgtagaatatatatatatatatatatatttttttttttttctgggatatttctcctttcttctgttcattaatgtttttcatctatcattgtgttttaaatatgacctaaggttctatagtatatttagacaaacttctcatttctctgcagtgttcggaattcatcatgcctgtatgacaaggttgtgtttgagaacaaaaggggacctgtgtgacatgttttatttcaatatacatttagagtttgaacaaataaaaaaagctattagaatttttaatatatataacacattatcaaaaacactgtcacttttctgagtgctctaatcaggcaattcgtatgtatatttaaaaagagggaaaaagctaattagtatactaaatatatttattttaatacctgtgctcatatgttatctttaaagaacaggaataaaatttatgggtagaatagtatgccagcaatttaccttcaatacaaattaaacacatcttttaagaaaagggcgagttatattggctggggtggcactaacactacattattaatatgtatttattgagcatttactgtgtccaggaccctgtaagggggaaaaaaagtccttgtttttacaatctttattgactgaaattctgtaaaatgaattgaattcctgaagtgacatcatttaacccaaattttttcatcatcaactaattcatctcagaaaacagtaaagtttaaaaaaaaatactatcagtaagaataatatatttccttggtttggaaatttccaaaccacgatattctgccctgtcaaaatatattcactggggaattttttacttcatacgtttcttttgaacttagtcacatttttatatctagctggtttcattattaaataaaagaaaaggtgattcccctcttgtactgcaggagaccacatacttgaatgatactctaacttctaggttctgttatagtaacactgaaatcagaaacctgtgggagaaatgtcactttatctgagagagtaaaatcagactctaaaaggaagtagcaaattcatcttgttcttttttttaatcaaccctctgcctaagacaagtagtttgaaacacagaggactctttaagtcatacttcctttatgtagtcaaaaccaaagccactgggtagtgctttccccaaaggaatctctaaatagtagacggggacattttcagatagattcgtttgtaggcaaacctccattgcttgtatcacatttcctgaaagaataaaggtaaaacttcaactatgtattacagaaagaaaaattcagcctgaaccctacccttataaaacaggttaattgggttttaattttcataaatcataaaggactattttgaacatttgggcctttaattgtctagctcctagatgaagtacaaatcagaaaaaaaaaaaactgtactgtgtcagaatgcaagctttcctctttgcattttggcatttgaaaactccgaagagcggtttttgttttttatttaaagaagatgatacatatgtgtacccgattcaaaactagagaatagaatttaaaacataattttcaaagtcttcaaatatgcctaaaggtaacaatgtcatcttttaattgccaatttctctaccactttcaaaaaattacttccaaggatttaatgagctccttcctttcaacagaaaatggactattttcctttcagatttactatatgctgtcactccagctttataaccgcatgtgcatacacaaacatttctttctctcttgcaggtggcacaaaccaggaaggggaaatctgtggtttaaattctttatgcctcatcctctgagtgctgaaggcttgctgtaggctgtatgctgttaatgctaatcgtgataggggtttttgcctccaactgactcctacatattagcattaacagtgtatgatgcctgttactagcattcacatggaacaaattgctgccgtgggaggatgacaaagaagcatgagtcaccctgctggataaacttagacttcaggctttatcatttttcaatctgttaatcataatctggtcactgggatgttcaaccttaaactaagttttgaaagtaaggttatttaaaagatttatcagtagtatcctaaatgcaaacattttcatttaaatgtcaagcccatgtttgtttttatcattaacagaaaatatattcatgtcattcttaattgcaggttttggcttgttcattataatgttcataaacacctttgattcaactgttagaaatgtgggctaaacacaaatttctataatatttttgtagttaaaaattagaaggactactaacctccagttatatcatggattgtctggcaacgttttttaaaagatttagaaactggtactttcccccaggtaacgattttctgttcaggcaacttcagtttaaaattaatacttttatttgactcttaaagggaaactgaaaggctatgaagctgaatttttttaatgaaatatttttaacagttagcagggtaaataacatctgacagctaatgagatattttttccatacaagataaaaagatttaatcaaaaaatttcatatttgaaatgaagtcccaaatctaggttcaagttcaatagcttagccacataatacggttgtgcgagcagagaatctacctttccacttctaagcctgtttcttcctccatatggggataatactttacaaggttgttgtgaggcttagatgagatagagaattattccataagataatcaagtgctacattaatgttatagttagattaatccaagaactagtcaccctactttattagagaagagaaaagctaatgatttgatttgcagaatatttaaggtttggatttctatgcagtttttctaaataaccatcacttacaaatatgtaaccaaacgtaattgttagtatatttaatgtaaacttgttttaacaactcttctcaacattttgtccaggttattcactgtaaccaaataaatctcatgagtctttagttgatttaaaataa